(19) United States
(12) Patent Application Publication
(10) Pub. No.: US 2019/0307959 A1
(43) Pub. Date: Oct. 10, 2019

(54) AUTOINJECTOR

(71) Applicant: Nuance Designs of CT, LLC, Woodbridge, CT (US)

(72) Inventor: Carlos Guillermo, Atascadero, CA (US)

(73) Assignee: Nuance Designs of CT, LLC, Woodbridge, CT (US)

(21) Appl. No.: 16/380,733

(22) Filed: Apr. 10, 2019

Related U.S. Application Data
(60) Provisional application No. 62/821,865, filed on Mar. 21, 2019.
(60) Provisional application No. 62/821,262, filed on Mar. 20, 2019.
(60) Provisional application No. 62/655,387, filed on Apr. 10, 2018.

(51) Int. Cl. A61M 5/20; A61M 5/32

(52) U.S. Cl. CPC A61M 5/2053; A61M 2005/2086; A61M 2205/583; A61M 5/3204

(57) ABSTRACT

An autoinjector is provided that includes an actuator assembly having an actuator body and a relief valve assembly. The actuator body includes a receptacle, an open distal end in fluid communication with the receptacle, and a channel in fluid communication with the receptacle. The relief valve assembly includes a first timing member and a second timing member. The first timing member is movable between a primary position and a secondary position under the influence of a force. The second timing member is movable between a first position directly engaged with the first timing member and a second position spaced from the first timing member.

### CROSS-REFERENCE TO RELATED APPLICATIONS

This application claims the benefit of U.S. Provisional Application No. 62/821,865, filed Mar. 21, 2019 entitled “AUTOINJECTOR,” U.S. Provisional Application No. 62/821,262, filed Mar. 20, 2019 entitled “AUTOINJECTOR,” and U.S. Provisional Application No. 62/655,387, filed Apr. 10, 2018 entitled “AUTOINJECTOR,” the entire disclosures of each of which are incorporated by reference herein in their entirety.

### BACKGROUND OF THE INVENTION

The present invention relates generally to drug delivery devices. Specifically, the present invention relates to an autoinjector for delivering a drug to a patient.

An autoinjector is a medical device designed to deliver one or more doses of a particular drug in a manner that facilitates self-administration of the drug via a hypodermic needle. Autoinjectors were originally designed for military use to counteract nerve-agent poisonings. The devices later moved into the civilian realm, with the first civilian devices being introduced in the mid to late 1970s, to dispense epinephrine to treat anaphylaxis. More recently, these devices have seen broadened use.

By design, autoinjectors are easy to use and are intended for administration by patients to themselves, or by untrained personnel. Thus, they are typically self-contained and designed to require only a few basic steps to operate.

Typically, autoinjectors are spring-actuated. This means that one or more springs are used to drive the drug through the needle of the autoinjector, and in some cases, to insert the needle into the patient as well. At least one spring is typically used to apply a force to a stopper of a syringe or cartridge, much in the manner that a person would manually actuate a syringe plunger, and drive the drug out of the syringe into the injection site. These autoinjectors typically deliver a full dose of their drug in about 5 to 10 seconds.

An alternative form of autoinjector is the gas jet injector, which dispenses with a needle entirely, instead using a high-pressure narrow jet of the drug itself to penetrate the skin. Gas jet injectors have predominantly been used for mass vaccinations, not single dose delivery, and involve delivery of the drug at pressures of about 4,000 psi almost instantaneously. Newer gas jet injectors use slightly lower pressures. In general however, gas jet injectors are limited in volume they can deliver in a single “shot” and the depth to which they can deliver the drug. In addition, as an explosive/high impact technology, they cause impact and jarring that can be problematic.

Current designs in spring-actuated autoinjectors involve making tradeoffs among various controllable and uncontrollable factors to insure reliable, proper and complete dose delivery. However, the selected tradeoffs that provide for reliable, proper and complete dose delivery can result in the inability to provide certain desirable feature(s) or requiring of greater complexity to provide less than desirable version(s) of such feature(s).

### BRIEF SUMMARY OF THE INVENTION

In accordance with an exemplary embodiment, the subject disclosure provides a power pack for an autoinjector that includes an actuator assembly for mounting to an autoinjector. The actuator assembly includes an actuator body and a relief valve assembly mounted to the actuator body. The actuator body has a receptacle for receiving a canister, an open distal end in fluid communication with the receptacle, and a channel in fluid communication with the receptacle. The relief valve assembly includes a first timing member and a second timing member. The first timing member is movable between a primary position and a secondary position under the influence of a force. The second timing member is movable between a first position directly engaged with the first timing member and a second position spaced from the first timing member.

The power pack further includes a canister having a pressurized driver mounted within the receptacle, and a regulator mounted within the receptacle. The actuator body further includes a recess for receiving the relief valve assembly, and the recess is in fluid communication with the channel. The power pack is further configured whereby, when in the primary position, the first timing member locks the second timing member in the first position. The relief valve assembly houses a damping element engaging the first timing member. The damping element is a viscous fluid. The relief valve assembly further includes a reservoir chamber housing the damping element.

The subject disclosure further provides an autoinjector including a housing, the power pack discussed above mounted within the housing, and a medicament container mounted within the housing and operatively connected to the power pack and in fluid communication with the open distal end of the actuator body.

In accordance with another exemplary embodiment, the subject disclosure provides a power pack for an autoinjector that includes an actuator assembly for mounting to an autoinjector. The actuator includes an actuator body. The actuator body has a first housing for receiving a canister, and a second housing in fluid communication with the first housing. The first housing has an open distal end. The second housing includes a first opening in fluid communication with an exterior of the actuator body and a second opening in fluid communication with the exterior of the actuator body and spaced from the first opening.

The power pack further includes a canister having a pressurized driver mounted within the first housing, a regulator mounted within the first housing, and a relief valve assembly mounted within the second housing. The relief valve assembly includes a first timing member and a second timing member. The first timing member is movable between a primary position and a secondary position. The second timing member is movable between a first position sealing the first opening and a second position spaced from the first opening. The relief valve assembly further includes a reservoir chamber housing a damping element engaging the first timing member. The damping element is a viscous fluid. The reservoir chamber housing sealingly engages the second opening. The first timing member moves along an axis substantially transverse to an axis along which the second timing member moves. The power pack is further configured whereby, when in the primary position, the first timing members locks the second timing member in the first position.

The subject disclosure further provides an autoinjector including a housing, the power pack discussed above mounted within the housing, and a medicament container mounted within the housing and operatively connected to the power pack and in fluid communication with the open distal end of the actuator body.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the exemplary embodiments are not limited to the precise arrangements and instrumentalities shown.

In the drawings:

FIGS. 1A-1E are various views of an exemplary embodiment of an autoinjector in accordance with the subject disclosure;

FIG. 2A is a longitudinal cross-sectional view of the autoinjector of FIG. 1A taken along a midline plane;

FIG. 2B is another longitudinal cross-sectional view of the autoinjector of FIG. 1A taken along a plane transverse to the midline plane of FIG. 2A;

FIG. 3 is a perspective view of the autoinjector of FIG. 1A with a housing component omitted for purposes of illustration;

FIG. 4 is a perspective view of an autoinjector shield component of the autoinjector of FIG. 3;

FIG. 5 is a perspective view of the autoinjector of FIG. 1A with a housing and an autoinjector shield component omitted;

FIG. 6 is a perspective view of a cradle component of the autoinjector of FIG. 5;

FIG. 7 is a perspective view of the autoinjector of FIG. 1A with certain components omitted for purposes of illustration;

FIG. 8 is a perspective view of a power pack of the autoinjector of FIG. 1A;

FIGS. 9A-H are various views of an actuator body of the autoinjector of FIG. 1A;

FIGS. 10A-B are a top and a bottom perspective view of a first timing member of the autoinjector of FIG. 1A;

FIGS. 11A-B are various views of a second timing member of the autoinjector of FIG. 1A;

FIGS. 12A-C are various views of a fluid reservoir of the autoinjector of FIG. 1A;

FIGS. 13A-C are various views of a plunger of the autoinjector of FIG. 1A;

FIGS. 14A-B are top and bottom perspective views of a regulator of the autoinjector of FIG. 1A;

FIGS. 15A-B are a perspective view and a longitudinal cross-sectional view of an actuator assembly of the autoinjector of FIG. 1A in an initial state with certain components omitted and/or in phantom for purposes of illustration;

FIGS. 16A-B are side and longitudinal cross-sectional views of an actuator assembly of the autoinjector of FIG. 1A in an armed state;

FIGS. 17A-B are side and longitudinal cross-sectional views of an actuator assembly of the autoinjector of FIG. 1A in an end-of-dose state;

FIG. 18 is a longitudinal cross-sectional view of an actuator assembly of the autoinjector of FIG. 1A in a retracted state;

FIGS. 19A-E are various views of an alternative exemplary embodiment of an autoinjector in accordance with the subject disclosure;

FIG. 20A is a longitudinal cross-sectional view of the autoinjector of FIG. 19A in an initial capped state taken along a midline plane;

FIG. 20B is a longitudinal cross-sectional view of the autoinjector of FIG. 19A in an initial capped state taken along a plane transverse to the midline plane of FIG. 20A;

FIG. 20C is a longitudinal cross-sectional view of the autoinjector of FIG. 19A in an armed state taken along a midline plane;

FIG. 20D is a longitudinal cross-sectional view of the autoinjector of FIG. 19A in an injecting state taken along a midline plane;

FIG. 20E is a longitudinal cross-sectional view of the autoinjector of FIG. 19A in a retracted state taken along a midline plane;

FIG. 21 is a perspective view of the autoinjector of FIG. 19A with certain components omitted for purposes of illustration;

FIGS. 22A-H are various views of an actuator body of the autoinjector of FIG. 19A;

FIGS. 23A-B are various views of a second timing member of the autoinjector of FIG. 19A;

FIGS. 24A-C are various views of a fluid reservoir of the autoinjector of FIG. 19A;

FIG. 25A is a perspective view of the autoinjector of FIG. 19A with certain components omitted or in phantom for purposes of illustration;

FIG. 25B is an enlarged partial perspective view of a proximal end of the autoinjector shown in FIG. 25A;

FIG. 25C is a perspective view of the autoinjector of FIG. 19A with certain components omitted or in phantom for purposes of illustration; and

FIGS. 26A-F are various views of an activator of the autoinjector of FIG. 19A.

### DETAILED DESCRIPTION OF THE INVENTION

Reference will now be made in detail to the exemplary embodiments of the autoinjector illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import.

Certain terminology is used in the following description for convenience only and is not limiting. The words “right,” “left,” “upper,” and “lower” designate directions in the drawings to which reference is made. The terminology includes the words above specifically mentioned, derivatives thereof, and words of similar import. With reference to an autoinjector, the “distal end” of the autoinjector refers to the end of the autoinjector towards the needle while the “proximal end” of the autoinjector refers to the end of the autoinjector towards the actuator assembly.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the invention may be combined in any suitable manner in one or more embodiments. One skilled in the relevant art will recognize, in light of the description herein, that the invention can be practiced without one or more of the specific features or advantages of a particular exemplary embodiment. In other instances, additional features and advantages may be recognized in certain exemplary embodiments that may not be present in all exemplary embodiments of the invention.

For ease of explanation, as used herein, the term “syringe” means any combination of a drug-containing container, a hypodermic needle and a pathway between the two through which the drug can be delivered from the container into a living body via the hypodermic needle, irrespective of the relative proximity between the container and needle themselves. Representative, specific examples of “syringes” as defined herein include (but are not intended to be limited to): conventional staked needle syringes, prefilled syringes, removable hub needle/syringe body systems including those with a luer taper, infusion sets, single use and multi-use cartridge-based syringe systems, multi-chambered and variable dose syringes, as well as cartridges, vials and pouches (rigid or collapsible), and a drug to be used in conjunction with a needle to deliver an injection volume (i.e., a dose) of the drug.

Similarly, the use of the term “autoinjector” herein is intended to encompass both the conventional understanding of that term, as well as any other small form factor, hand-holdable or wearable, injection-type, or infusion-type (i.e., for delivery of a drug via a needle over a period of time lasting on the order of several minutes) drug delivery device.

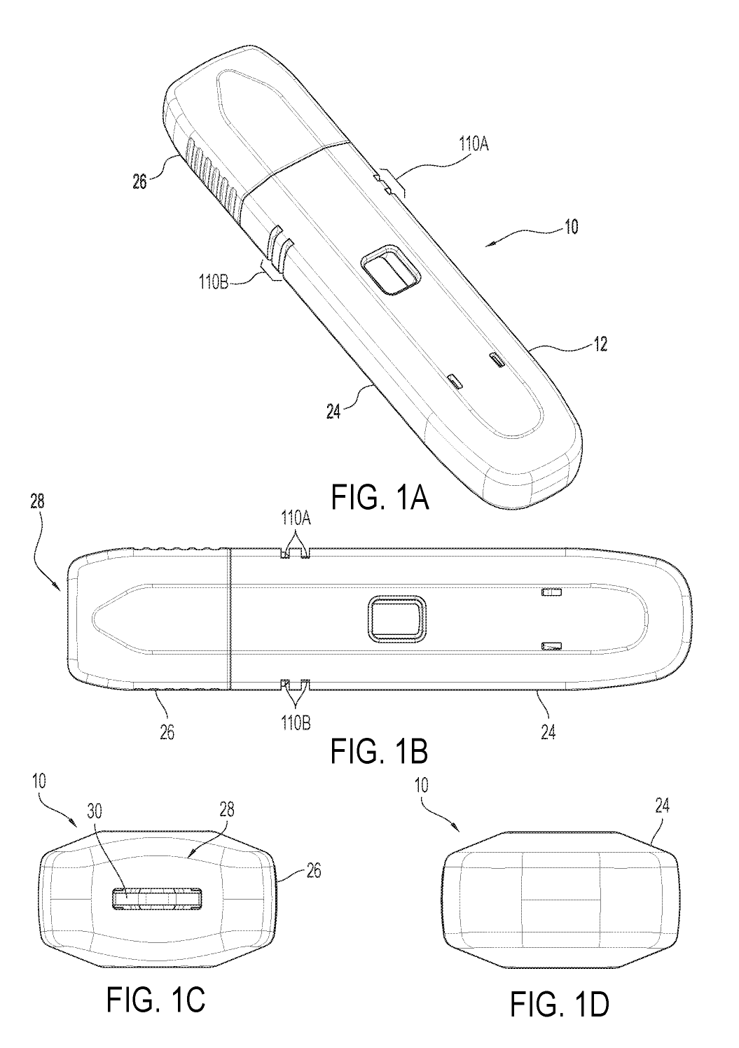

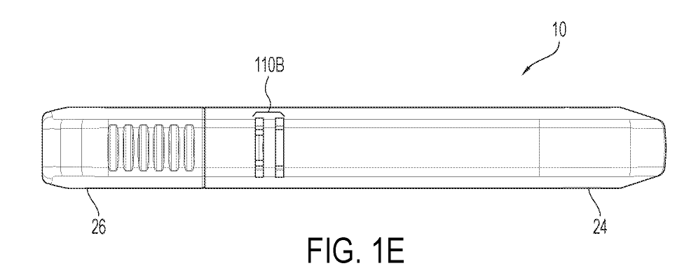

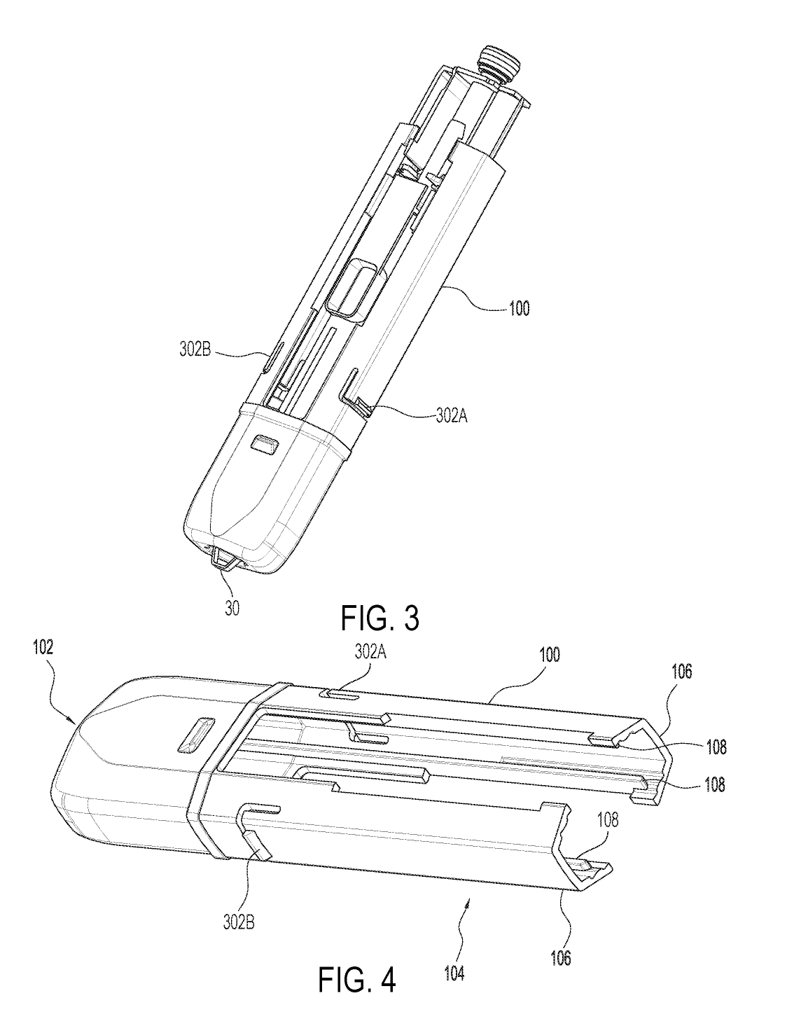

Referring to FIGS. 1-18 there is shown an exemplary embodiment of an autoinjector 10 in accordance with the subject disclosure. The autoinjector includes a housing 12, a syringe 14, and an actuator assembly 1500.

In accordance with an exemplary aspect, the syringe 14 can be a staked needle syringe that includes a needle 18, a syringe barrel 20 having a lip 22, and a stopper or piston 25. The syringe 14 is housed within the housing 12 and movable relative to the housing, as further discussed below.

The housing 12 is configured as best shown in FIG. 1. The housing includes a main body housing 24 to be grasped by a user's hand and a removable housing section 26 about a distal end 28 of the autoinjector. The removable housing section is configured as a cap, designed to be removed by the user for exposing the distal end of the autoinjector.

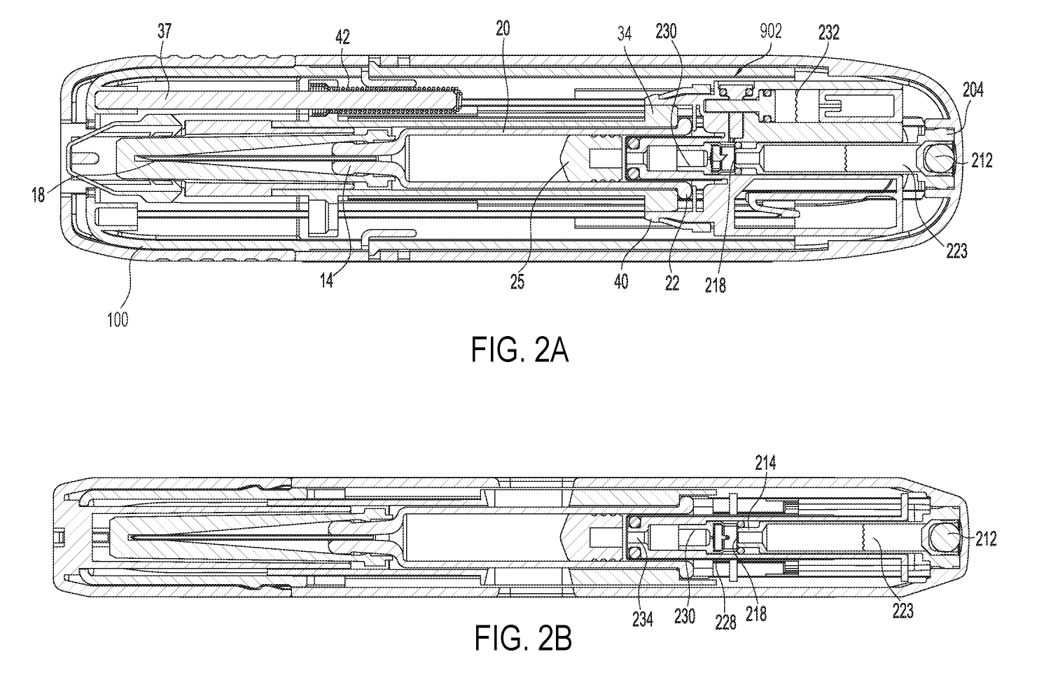

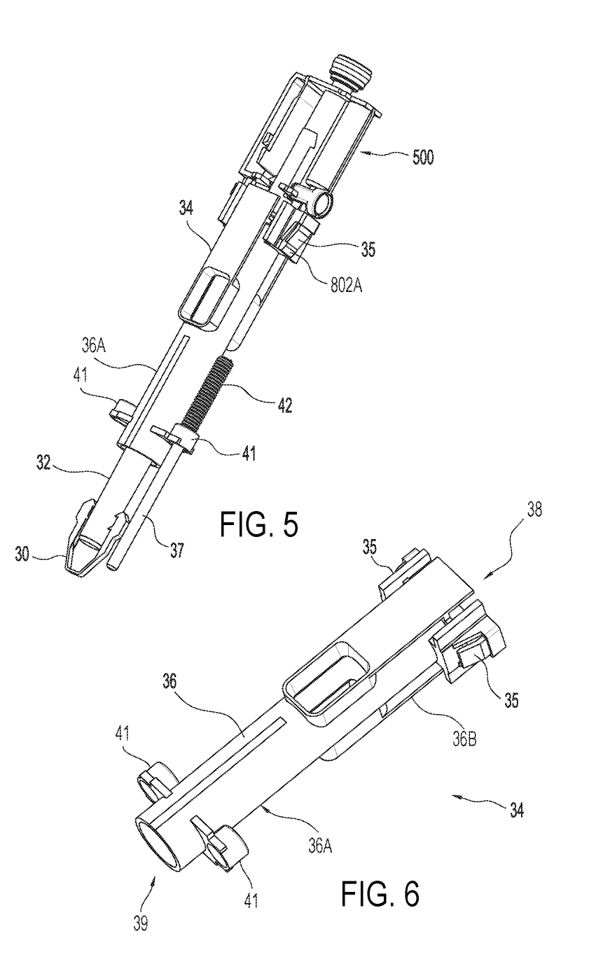

Referring to FIGS. 2A and 5, fixedly mounted within the removable housing section 26 is a gripping member or clamp 30 for engaging a needle shield 32 of the syringe. The gripping member 30 is configured as best shown in FIG. 5 as a clamp-like gripper having diametrically opposed arms with inwardly facing gripping ridges to facilitate gripping of the needle shield.

Referring to FIG. 5, the needle shield 32 can be configured as any conventional needle shield for shielding the syringe needle 18. The needle shield is releasably mounted to the syringe 14. The needle shield can optionally include ridges (not shown) on its outer surface to facilitate gripping by the gripping member. Upon separation of the removable housing section 26 from the main body housing 24, the gripping member 30 retains the needle shield so as to withdraw it from a cradle 34 (FIG. 6).

Referring to FIGS. 1A, 1B, 1E, 3, and 4, the autoinjector shield 100 is moveable relative to the syringe. The autoinjector shield includes a distal end 102 for shielding the syringe needle and a proximal end 104. The autoinjector shield is connected to the housing 12 by cooperating tabs or catches 302A, 302B on the autoinjector shield and slots 110A, 110B on the housing. Specifically, the distal end includes cooperating tabs 302A, 302B for engaging with cooperating slots 110A, 110B on the housing (FIGS. 1B, 1E, 2A, and 4). FIGS. 2A and 2B illustrate cross-sectional views of the housing 12 connected and assembled to the cradle 34 and autoinjector shield 100. About a proximal end of the shield are spaced apart legs or latch members 106 having an elongated opening extending therebetween for receipt of the cradle 34 and the actuator body 900. Each leg includes an extended slat 108 for engaging a cantilever or hook 808 of the actuator body 900, as further discussed below.

The cradle 34 is configured as best shown in FIGS. 2A and 6. The cradle is coupled to the syringe or medicine cartridge 14 and movable relative to the autoinjector shield 100. The cradle is configured as a substantially cylindrical body 36 having an interior sized sufficiently to house the syringe, an open proximal end 38, and a distal end 39. The proximal end includes a flange for engaging the lip 22 of the syringe barrel 20. The cradle is also sized to have a longitudinal length sufficient to allow a majority of the needle 18 to extend outwardly from its distal end. The cradle further includes one or more mounts 41 for receiving a pin 37 and a retraction biasing member 42, as described in further detail below.

Referring to FIGS. 5, 6, and 8, the cradle 34 is mounted to the actuator body 900 via cooperating catches 35 on the cradle and cooperating catches 804 on the actuator body. The cooperating catches 35, 804 engage one another to hold the cradle relative to the actuator assembly.

Referring to FIGS. 2A and 5, the distal body 36A of the cradle 34 retains the retraction biasing member 42 positioned along its lateral side. The retraction biasing member includes a distal end engaged with the distal body. Specifically, the retraction biasing member engages or extends from the mount 41 about a lateral side of the distal body. The mount 41 has a distal end opening with a diameter larger than a proximal end opening. The proximal end opening of the mount is sized to be smaller than a distal end of the retraction biasing member. That is, the retraction biasing member 42 is configured, e.g., as a compression or extension spring, having a main body portion of substantially constant diameter, a distal end having a larger diameter than the main body portion, and a proximal end having a diameter smaller than the main body portion. As such, the retraction biasing member is sized and configured to pass through the mount but retained therein as a result of its larger diameter distal end that is sized to be larger than the proximal end opening of the mount.

The pin 37 and the retraction biasing member 42 are sized and configured such that the pin is slidable through the mount 41 and received within the main body portion of the retraction biasing member. Further, the pin diameter is sized to be greater than the proximal end of the retraction biasing member so that the pin does not pass through the proximal end of the retraction biasing member, but instead is contained therein. Thus, as the cradle 34 is moved distally relative to the autoinjector shield 100, the pin forces the retraction biasing member to elongate, thereby having the retraction biasing member apply a counterforce to bias the cradle proximally. As a result, the retraction biasing member biases the cradle in the proximal direction, e.g., upon completion of an injection by the autoinjector, or complete venting of pressurized gas within the drive chamber, thereby moving the actuator assembly proximally relative to the autoinjector shield, and/or retracting the syringe fully within the autoinjector shield.

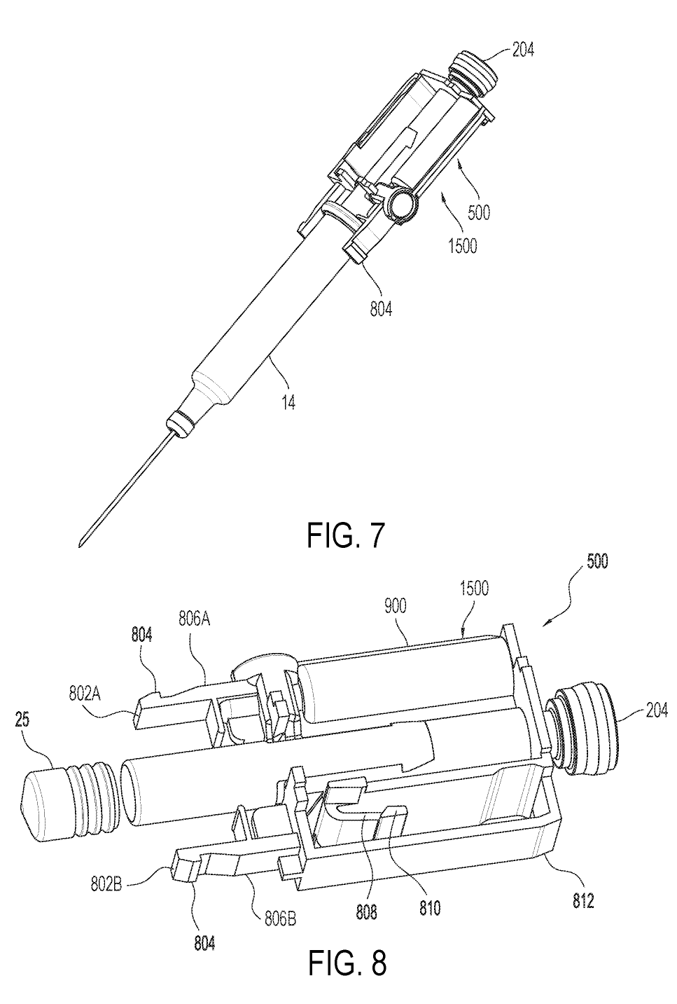

In accordance with an exemplary embodiment, the power pack 500 is configured as shown in FIGS. 7 and 8. The power pack includes an actuator assembly 1500, a canister 204 having a pressurized driver 223, and a regulator 1400.

The actuator assembly 1500 is configured as shown in FIGS. 2A, 2B, and 7-18. The actuator assembly includes an actuator body 900 and a relief valve assembly 902 mounted to the actuator body. The actuator assembly is configured for mounting to the autoinjector, as shown in FIG. 2A.

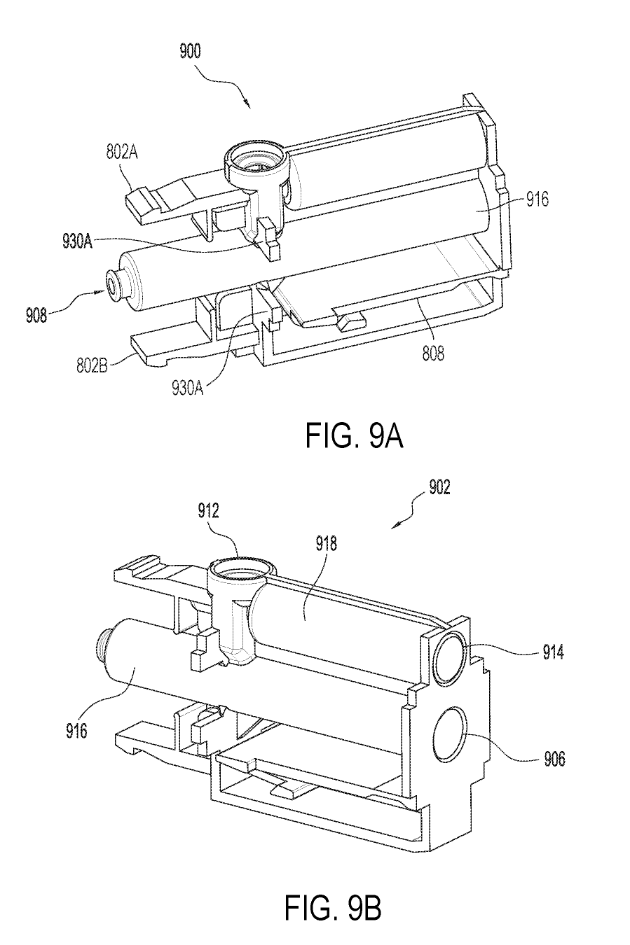

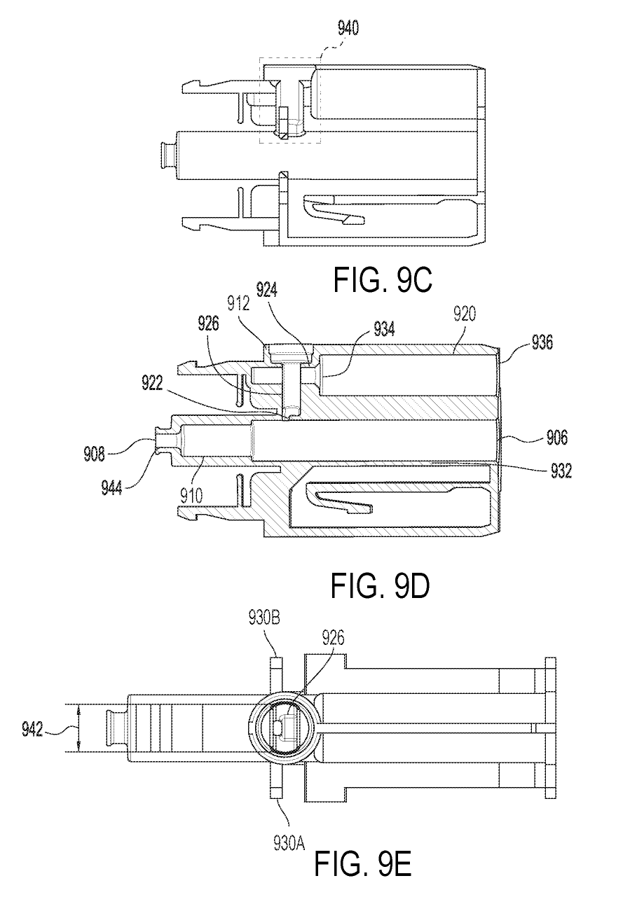

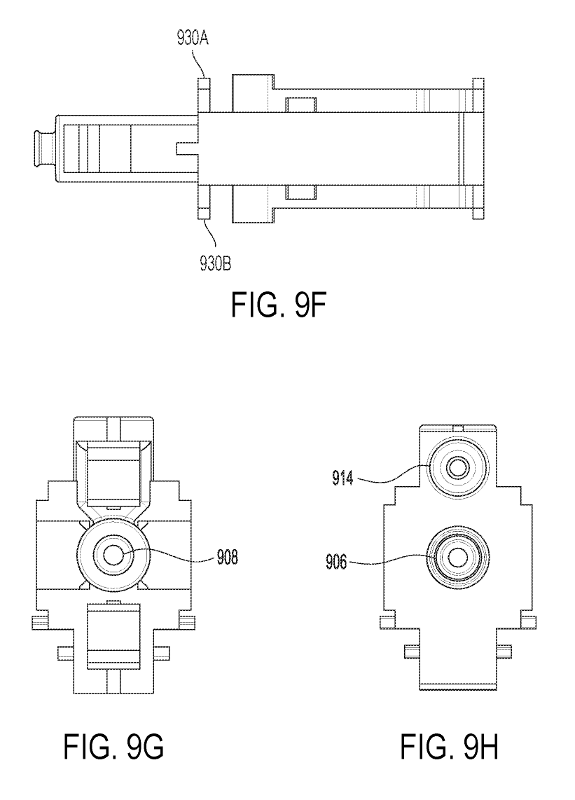

Referring to FIGS. 9A-H, the actuator body 900 is configured as a unitary part but can alternatively be configured as a multi-part component assembled or connected together. The actuator body includes a first housing 916, a second housing 918, first and second detents 802A, 802B, and a cantilever or hook 808.

In accordance with an exemplary aspect of the present embodiment, the first housing 916 is configured as a substantially cylindrical housing having an open proximal end 906 and an open distal end 908. However, the first housing can alternatively be configured as a non-cylindrical housing, such as a housing having a square, triangular, oval, or rectangular cross-sectional profile. The first housing also includes a larger diameter receptacle region 932 for receiving the canister 204 and a smaller diameter receptacle region 910 for receiving the flow regulator 230. The smaller diameter receptacle region has a diameter that is smaller than the larger diameter receptacle region and is distal to the larger diameter receptacle region. The smaller diameter receptacle region can be configured as a channel between the larger diameter receptacle region 932 and a neck 944.

The neck 944 extends distally from the smaller diameter receptacle region 910. The neck has an overall external and internal diameter smaller than the larger diameter receptacle region 932 and the smaller diameter receptacle region. The neck includes the open distal end 908, which in the present embodiment is a distally facing open end, but can alternatively be configured as a non-distally facing open end. For example, the open distal end can be configured as laterally facing openings or ports. The internal region defined by the neck is in fluid communication with the smaller diameter receptacle region and the larger diameter receptacle region.

The second housing 918 is adjacent the first housing 916 and includes a reservoir chamber 920 (FIG. 9D) and a relief valve chamber 940 (FIG. 9C). The reservoir chamber is configured as a substantially cylindrical housing, but alternatively can be configured as a housing having a non-cylindrical shape, e.g., a housing having a square, triangular, oval, or rectangular cross-section. The reservoir chamber has a longitudinal axis that is substantially parallel to a longitudinal axis of the first housing. In an exemplary aspect, a longitudinal length of the second housing is shorter than a longitudinal length of the first housing and/or the larger diameter receptacle region 932. The reservoir chamber includes an open proximal end 936 and an open distal end 934.

Referring to FIGS. 9C-E, the relief valve chamber 940 includes a first internal cavity 924 having a substantially cylindrical shape, and a second internal cavity 926. The second internal cavity is configured having a substantially rectangular shape, but alternatively can be configured as a cavity having a non-rectangular shape, e.g., a cavity having a cylindrical, oval, square, or triangular cross-section. The second internal cavity extends and has a longitudinal axis that traverses a longitudinal extent or longitudinal axis of the first internal cavity. A flow passageway 922 extends from the first housing 916 to the second internal cavity providing fluid communication from the first housing to the second internal cavity.

Extending laterally from the second internal cavity is a first opening 912 of the second housing. The first opening has a substantially cylindrical shape with a larger diameter opening than the second internal cavity. That is, the first opening in accordance with an exemplary aspect has a larger internal diameter than an internal rectangular cross-sectional area 942 of the second internal cavity. The first opening and second internal cavity are sized and shaped to receive the relief valve assembly 902, as further discussed below.

The first internal cavity 924 extends from the open distal end 934 of the reservoir chamber 920 and is in fluid communication with the reservoir chamber. The first internal cavity is also in fluid communication with the second internal cavity and is sized and shaped to receive the relief valve assembly 902 as further discussed below.

With reference to FIG. 9B, the second housing includes a second opening 914 about its proximal end. The second opening is a larger diameter opening than the first internal cavity 924 (FIG. 9D). That is, the second opening in accordance with an exemplary aspect has a larger internal diameter than an internal diameter of the first internal cavity of the relief valve chamber 940. The second opening and first internal cavity are sized and shaped to receive the relief valve assembly 902, as further discussed below.

Referring back to FIGS. 5, 6, and 8, the first and second detents 802A, 802B include arms 806A, 806B respectively that extend distally away from the actuator body. The arms include catches 804A, 804B that matingly engage cooperating catches 502A, 502B on the cradle 34.

Referring to FIGS. 2A, 4, and 8, the cantilever or hook 808 is a substantially J-shaped hook that extends from a proximal end of the actuator body 900 towards the distal end of the actuator body and is positioned adjacent the first housing 916. The cantilever includes tabs 810 that abut and engage the extended slats 108 of the autoinjector shield 100 (FIG. 4). The cantilever 808 is circumscribed by a wall 812. Alternatively, the tabs can be formed extending from any portion of the wall or first housing such that the tabs are positioned to engage the slats 108 of the autoinjector shield.

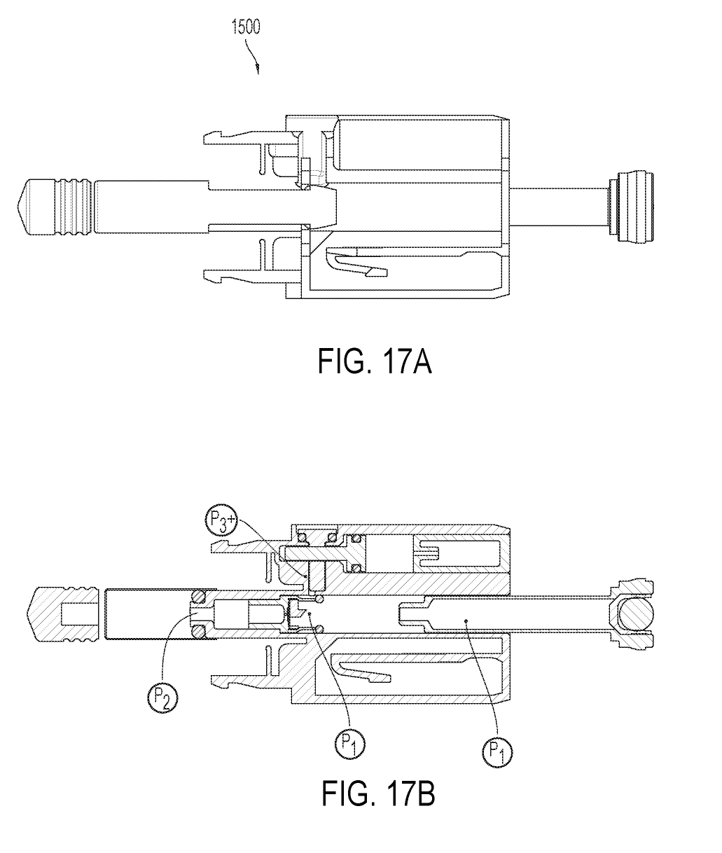

Referring to FIGS. 9A, 9E, 9F, and 17A, the dosage tabs 930A, 930B extend diametrically on either side of the autoinjector body 900. In accordance with an exemplary aspect, the dosage tabs 930A are formed as a pair of tabs along a first side of the first housing 916. The dosage tabs 930B are formed as a pair of tabs along a second side opposite the first side of the second housing. The dosage tabs 930A, 930B can be integrally formed with the actuator body.

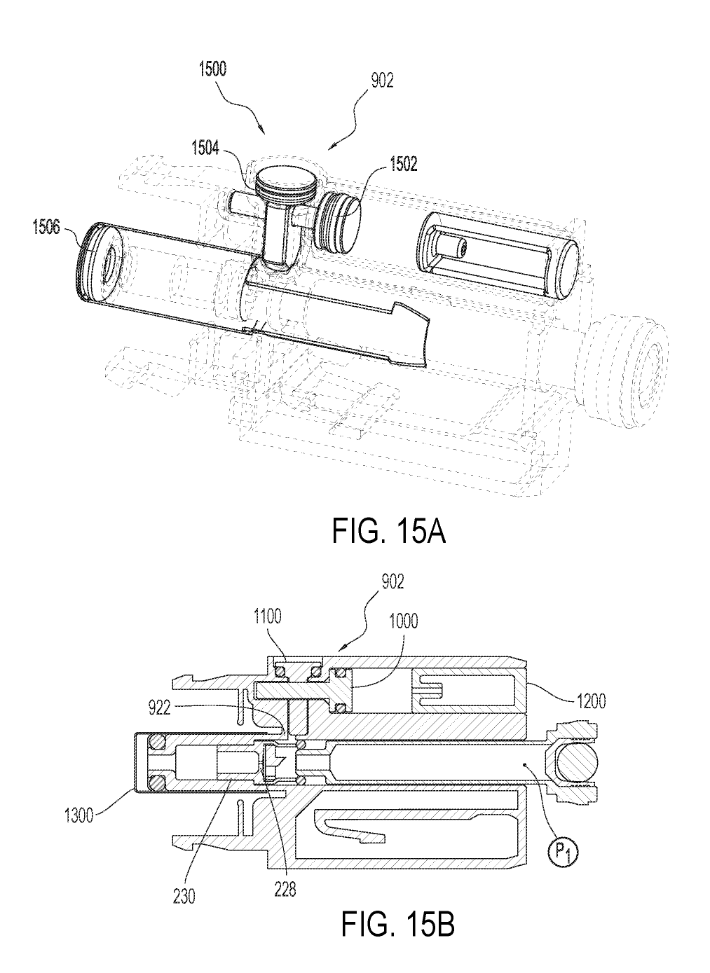

Referring to FIGS. 15A and 15B, the relief valve assembly 902 includes a first timing member 1000, a second timing member 1100, a fluid reservoir 1200, and a damping element 232 (FIG. 2A). The relief valve assembly is in fluid communication with the first housing 916 via the flow passageway 922. The relief valve assembly is configured to divert pressure build up from the first housing out towards an area external to the actuator body.

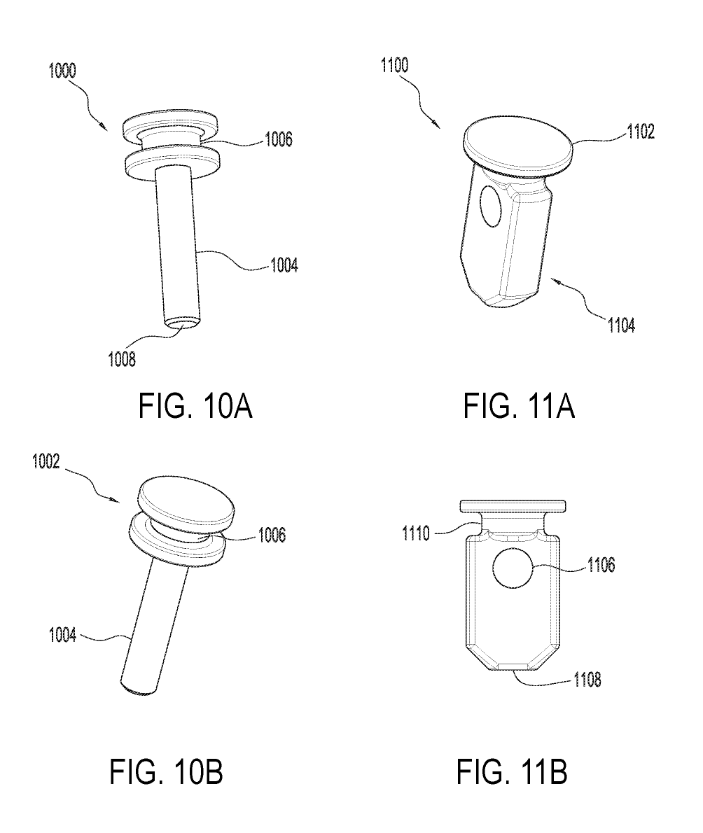

In accordance with an exemplary aspect of the present embodiment, the first timing member 1000 is configured as best shown in FIGS. 10A-B, and can be configured as a timing pin. The first timing member includes an increased diameter portion 1002 that is substantially circular and a reduced diameter portion 1004 that is substantially cylindrical extending distally from the increased diameter portion. The reduced diameter portion is sized and shaped to be inserted into, and conform to, the first internal cavity 924 of the relief valve chamber 940. That is, the reduced diameter portion conforms with the internal walls of the first internal cavity. The increased diameter portion 1002 is configured to slidably move within the reservoir chamber 920 of the second housing 918 from the open distal end 934 towards the open proximal end 936. The increased diameter portion conforms with the internal walls of the reservoir chamber.

The first timing member can also include an inner race 1006 that is sized and shaped for receiving a sealing member 1502 (FIG. 15A). The sealing member sealingly engages the reservoir chamber 920 so as to form a seal between the reservoir chamber 920 and the first internal cavity 924 of the relief valve chamber 940 while the first timing member is in the primary position. The sealing member may be an O-ring, or any other seal suitable for its intended purpose. In accordance with an exemplary aspect, the seal and O-ring may be selected so as to dampen or control the rate or timing of the motion of the first timing member, for example based on the material selected for the O-ring and/or the conforming fit to the inner diameter of the reservoir chamber.

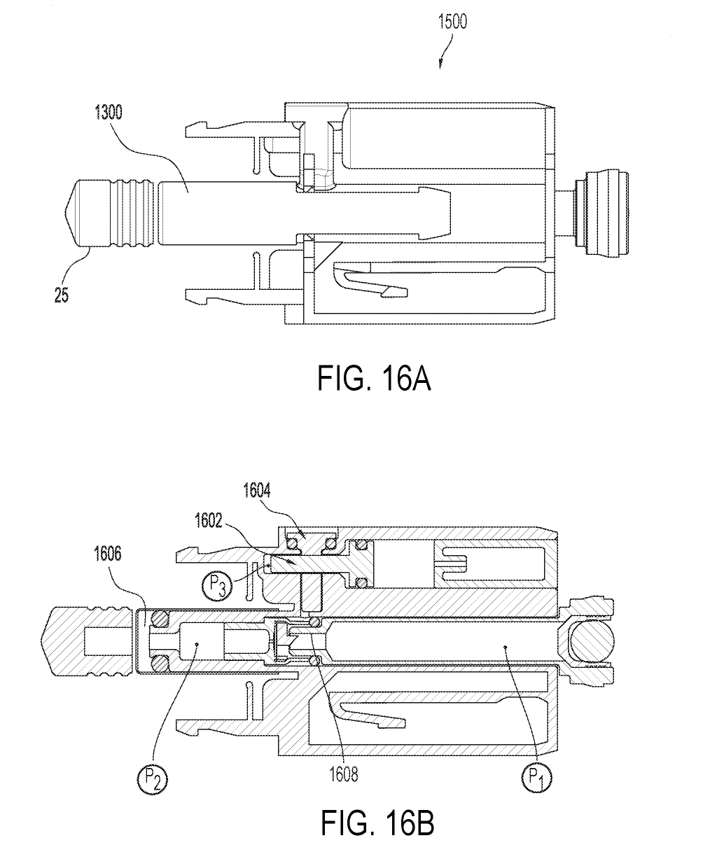

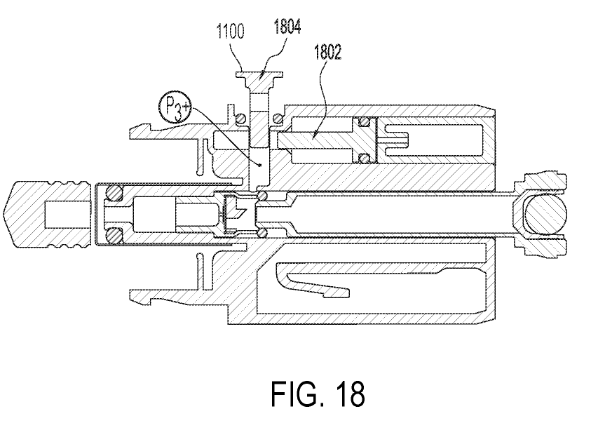

The first timing member 1000 is movable from a primary position 1602 (FIG. 16B) to a secondary position 1802 (FIG. 18) under the influence of a force. By way of non-limiting example, the force can include a pressure that is based on a pressure delivered from the canister 204. In the primary position, the first timing member locks the second timing member 1100 in its first position 1604 (FIG. 16B). In the secondary position, the first timing member is free of the second timing member, thereby leaving the second timing member unrestricted and able to move to its second position 1804 (FIG. 18B). When moving from the primary to the secondary position, the first timing member moves along an axis substantially transverse to an axis along which the second timing member moves.

In accordance with an exemplary aspect of the present embodiment, the second timing member 1100 is configured as best shown in FIGS. 11A-B and can be configured as a timing pin. The second timing member includes an increased width portion 1102 and a reduced width portion 1104. The increased width portion is substantially circular and configured to slidably move within the first opening 912. The reduced width portion is substantially rectangular and is sized and shaped to be inserted into, and conform to, the second internal cavity 926 within the relief valve chamber 940. That is, the reduced width portion conforms and/or sealingly engages the internal walls of the first opening. The reduced width portion includes a through hole 1106 extending completely therethrough. The through hole is sized and shaped to slidably receive and/or sealingly engage the reduced diameter portion 1004 of the first timing member 1000 therethrough.

The second timing member 1100 also includes an inner contour 1110. A sealing member 1504 (FIG. 15A) seats within the inner contour. The sealing member sealingly engages the first opening 912 so as to form a seal between the first opening and the neck 924 while the first timing member is in the primary position. By way of non-limiting example, the sealing member may be an O-ring, or any other seal suitable for its intended purpose.

The second timing member 1100 is movable from a first position 1604 (FIG. 16B) to a second position 1804 (FIG. 18B). In the first position, the second timing member is directly engaged with the first timing member 1000 and the second timing member seals the first opening 912. In the second position, the second timing member is spaced apart from the first timing member, thereby allowing pressurized driver to vent from the first housing 916 and out of the first opening.

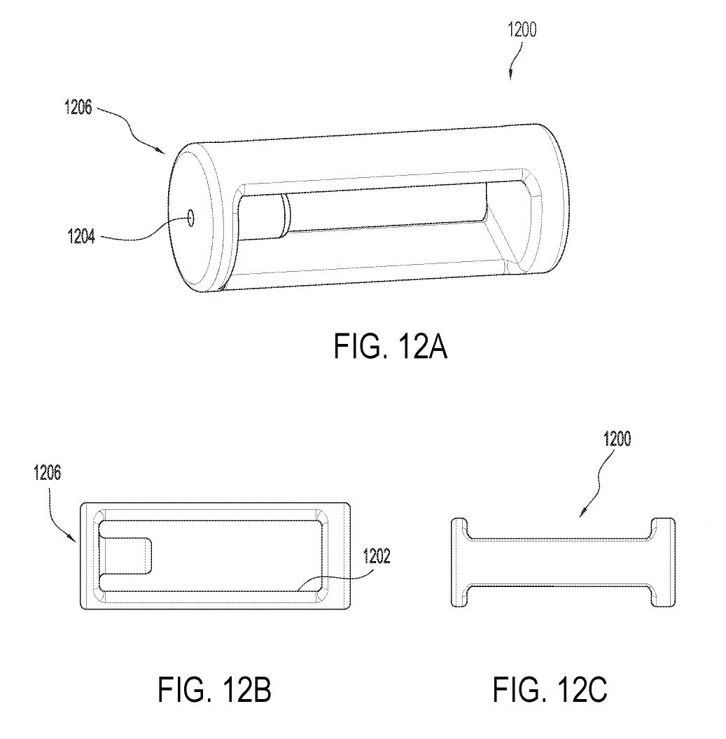

Referring to FIGS. 12A-C, the fluid reservoir or delay plug 1200 is substantially cylindrical and includes a cavity 1202 and an input 1204. The fluid reservoir is sized and shaped so as to be inserted into, and fixed in position within the reservoir chamber 920 of the second housing 918 (FIG. 9D). The distal end 1206 of the fluid reservoir includes the input defined as an aperture or opening. The input is operable for fluid communication between the cavity 1202 and the open distal end 934 of the reservoir chamber 920. Specifically, the input is operable to receive a damping element 232 (FIG. 2A) through the input and into the cavity, as the first timing member 1000 moves from the primary position to the secondary position. The input is sized to have an opening significantly smaller than a width of the reservoir chamber, such as 1%, 5%, 10%, 15%, 20%, or 25% of the width of the reservoir chamber such that the fluid flow of the damping element is restricted thereby providing resistance to movement of the first timing member.

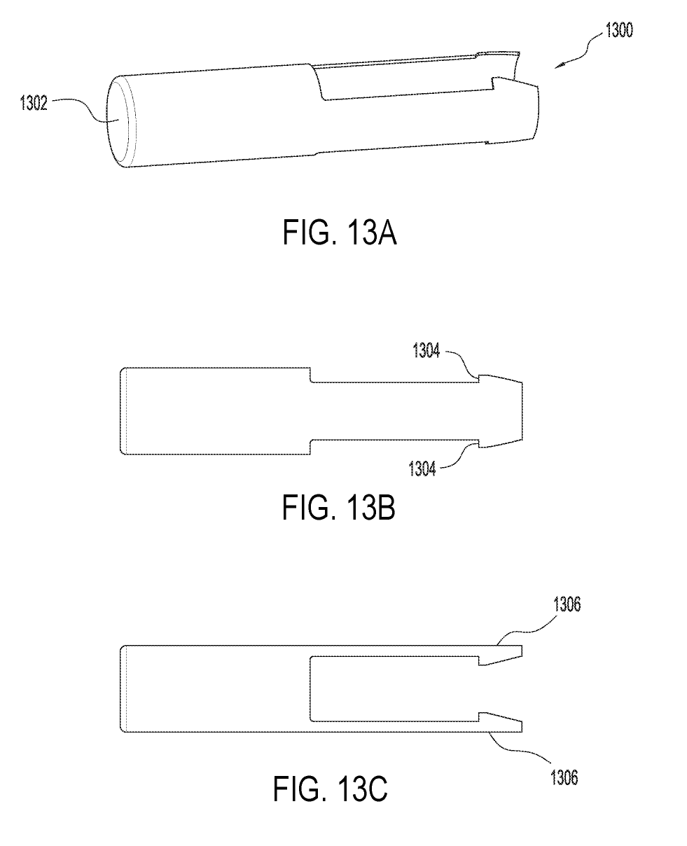

In accordance with an exemplary embodiment, the plunger 1300 is configured as best shown in FIGS. 13A-C. The plunger is configured as an intermediate member that slidably circumscribes the first housing 916 of the actuator body 900 (FIG. 15B). The plunger can be substantially cylindrical and includes a bottom 1302, one or more arms 1306, and one or more dosage stops 1304. The plunger is sized and shaped to be inserted into, and conform to, the interior of the syringe barrel. The bottom of the plunger is planar for engaging a corresponding proximal end of the stopper 25 when the plunger is actuated under the influence of a force exerted from pressurized gas released from the power pack. Although the plunger can include two arms, the plunger may alternatively include a single arm, or a plurality of arms, e.g., 3, 4, or more than 4 arms, for stopping the motion of the plunger when the plunger is actuated. The plunger is configured to contact the stopper 25 in a way that mimics a traditional syringe plunger rod at the stopper and thereby stabilizes and provides proper guidance for the stopper.

The stops 1304 may be configured as tabs that extend outwardly from the arms. The stops are configured to abut the dosage tabs 930 that extend outwardly from the actuator body 900 (FIG. 9A). The plunger enables a dose stop feature for precise dosing of only part of the syringe contents (for example, 0.3 mL from a 1 mL filled syringe).

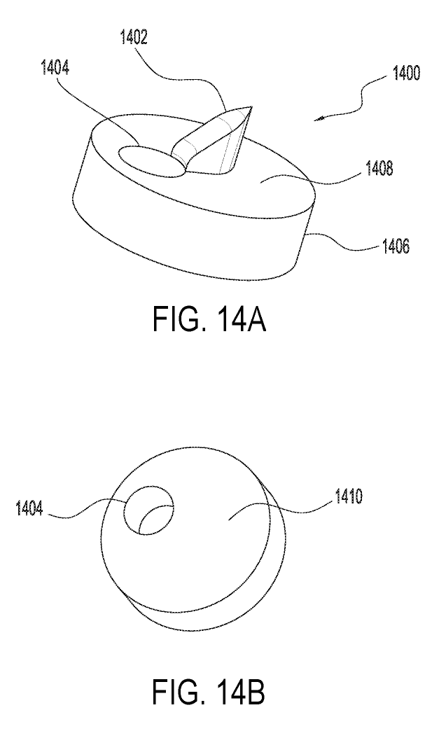

The regulator 1400 is configured as best shown in FIGS. 14A-B and situated within the distal end of the first housing of the actuator body (FIG. 15B). The regulator includes a substantially cylindrical body 1406 having a through hole 1404 extending through the body from its proximal surface 1408 to its distal surface 1410. Extending outwardly from its proximal surface is a piercing member 1402. The piercing member can be, for example, a cannula, a stake, a protuberance and the like that is sufficient to pierce a seal 218 of the canister 204. The piercing member is designed to engage and pierce the seal of the canister when the regulator is driven proximally toward the canister, or when the canister is driven distally within the actuator.

Alternative embodiments of a regulator applicable to the present autoinjector are described in U.S. Patent Application Publication Nos. 2014/0114248, 2014/0114250, and 2016/0361496, the entire disclosures of which are hereby incorporated by reference herein for all purposes.

Referring to FIG. 15B, the regulator 1400 is sized and shaped to be inserted into, and conform to, a proximal end of a regulator housing 230. The regulator housing has a proximal end with a larger diameter opening, a distal end with a smaller diameter opening, and a flow regulator 228. In accordance with an exemplary embodiment, the flow regulator 228 is configured as a through hole extending completely therethrough. The smaller diameter opening region has a diameter that is smaller than the larger diameter opening region and is distal to the larger diameter opening region. The flow regulator 228 is positioned so as to be in fluid communication with the regulator through hole 1404.

Referring to FIGS. 2A, 2B, and 9D, the canister 204 is mounted within the larger diameter receptacle region 932 of the actuator assembly 1500 and configured to be movable therein. The canister can be a substantially tubular canister having a tubular body 208, an openable proximal end, and a distal end 214. In accordance with an exemplary aspect of the present embodiment, the proximal end can be sealable by a plug 212. The plug can be configured as a sphere or cylinder that is sized and shaped to be inserted into an opening about the proximal end of the canister. In accordance with other alternative exemplary aspects, the proximal end can be crimped, or otherwise bonded or joined in a manner suitable for its intended purpose. The distal end has a neck 216 that is narrower than the tubular body and a sealed opening. In accordance with a further exemplary aspect, the opening can be covered by the pierceable seal 218. In accordance with alternative exemplary aspects, the seal can be integrated with the opening or the opening can otherwise be pierceably sealed in a manner suitable for its intended purpose.

The canister houses the driver 223 e.g., a propellant, or compressed or liquefied gas, that acts as a pressure source and is used to apply a force to a component of the actuator assembly 1500 or syringe 14, for example, the stopper 25, a rod, a cradle or other member, in a controlled manner, to thereby deliver the dose of drug or medicament from the syringe via the needle (e.g., by way of an injection). The force provided by the driver, in addition to providing a force for injection, can be used to provide some other form of action or motion e.g., timing, delay, retraction, insertion, indication, or other ancillary function of the autoinjector, as described in further detail below.

The canister can include a liquefied gas such as n-butane, nitrous oxide (N₂O) or carbon dioxide (CO₂) for delivering a flow of gas at a vapor pressure of P1. As used herein, “liquefied gas” is given its ordinary meaning, e.g. to refer to a gas that has been compressed to its vapor pressure so that an equilibrium exists within the vessel in which it is contained such that some portion of the volume is liquid. The pressure required for common liquefied gases at room temperature range from around 17 psi for n-butane, around 760 psi for nitrous oxide and around 850 psi for carbon dioxide. In addition, combinations of gasses can be used to modify the pressures to around a particular desired pressure. For example, specific hydrocarbon propellants (exemplary hydrocarbons can include but are not limited to butane, isobutane, and propane) can be mixed in varying quantities in a known manner to obtain pressures ranging from over about 17 psi to about 108 psi. Practically any pressure within the n-butane to carbon dioxide range can be obtained by mixing various gases having differing vapor pressures. A liquefied gas stored in a closed container has its internal pressure directly related only to its temperature and, for a fixed temperature, the pressure generally remains effectively constant until all the liquid portion has boiled off into the gaseous state. The use of a liquefied gas at the appropriate pressure in the manner described herein can provide advantages over conventional autoinjector technology because it allows for construction of an actuator assembly that can operate as a compact energy and constant pressure source. In addition, and advantageously, actuator assemblies can be constructed as described herein using a liquefied gas at a higher pressure than would be needed and regulate the pressure down to the desired use pressure. In doing so, advantages over conventional autoinjectors can be obtained. Other exemplary liquefied gases applicable to the exemplary autoinjector embodiments are described in U.S. Patent Application Publication Nos. 2014/0114248, 2014/0114250, and 2016/0361496.

The use of the term “compressed gas” as used herein means a gas that is stored at a pressure and temperature where the gas is never liquefied. With compressed gasses, as the gas is expelled from the container in which it is stored, the internal container pressure decreases. Common examples of such containers are SCUBA air tanks, which are commonly pressurized to around 3000 psi and compressed natural gas (CNG) tanks, which are commonly pressurized to about 2900-3600 psi. With compressed gasses, a pressure-regulating device must be used to obtain a constant pressure. In addition, because no liquefying occurs, the use of compressed gas is less desirable than liquefied gas because the container will tend to be larger and, due to the strength needed to contain the higher pressures, may be heavier as well. Compressed gas also loses pressure over time as it is being spent. In contrast, liquefied gas does not lose pressure over time but instead provides a constant pressure source.

Finally, as used herein, the terms “propellant,” “liquefied gas” or “compressed gas” are intended to also include gasses that may be the result of a chemical reaction within, or associated with, the storage container, in the instant example, the canister 204. Since the use of a particular “propellant,” “liquefied gas” or “compressed gas” will be implementation specific, as used herein, the term “driver” is intended to generically encompass “propellants,” “liquefied gases” and “compressed gases,” the selection of which will be a function of the particular intended implementation, and not mandated by the approach itself.

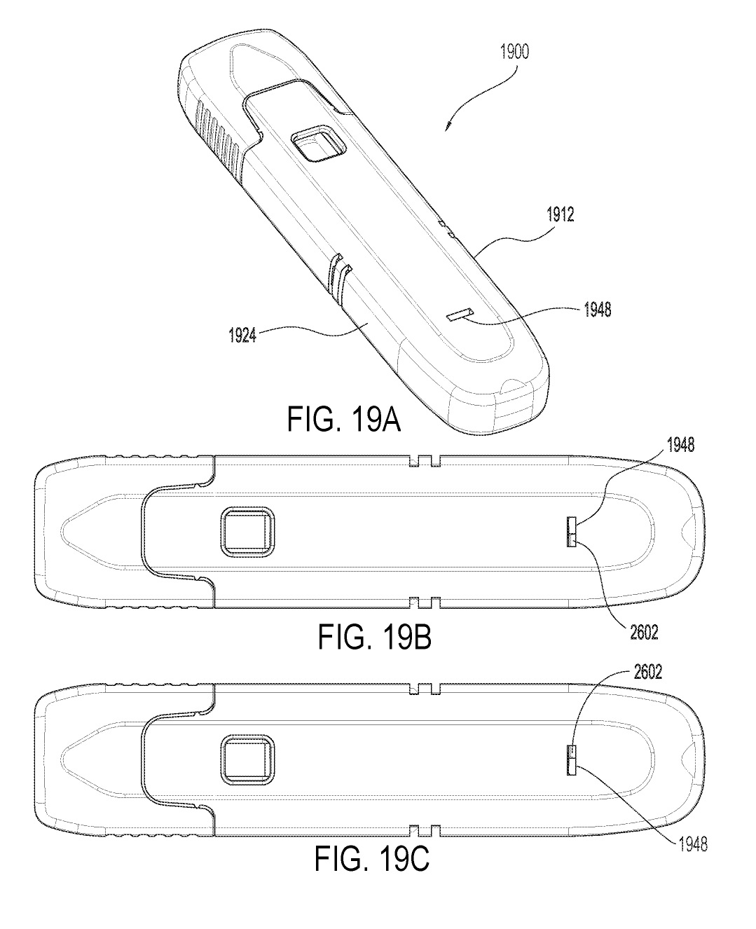

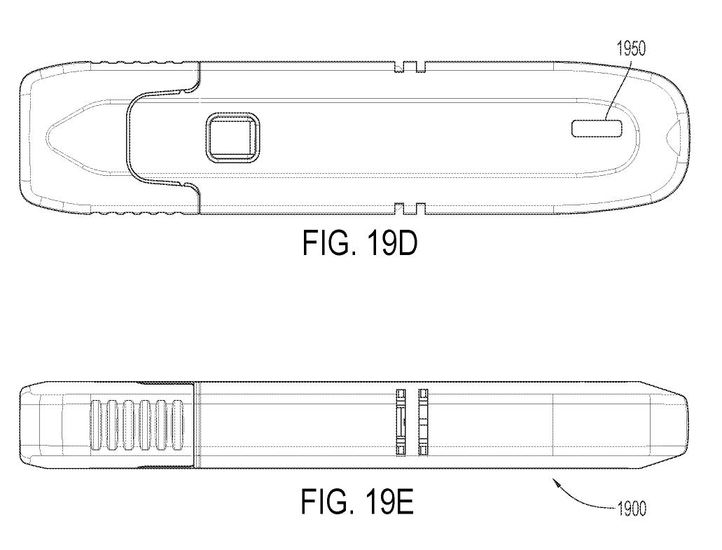

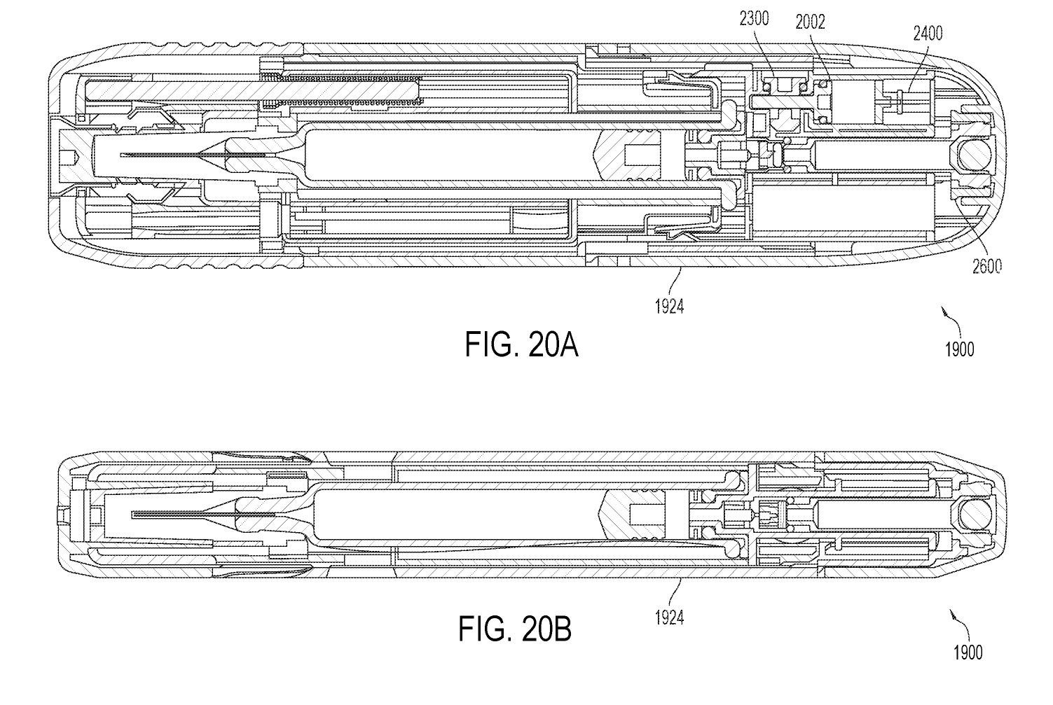

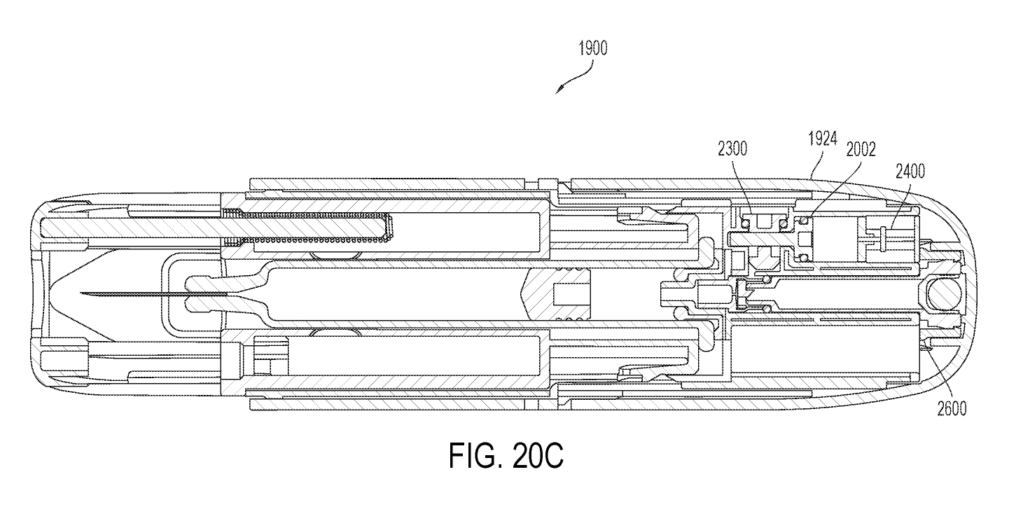

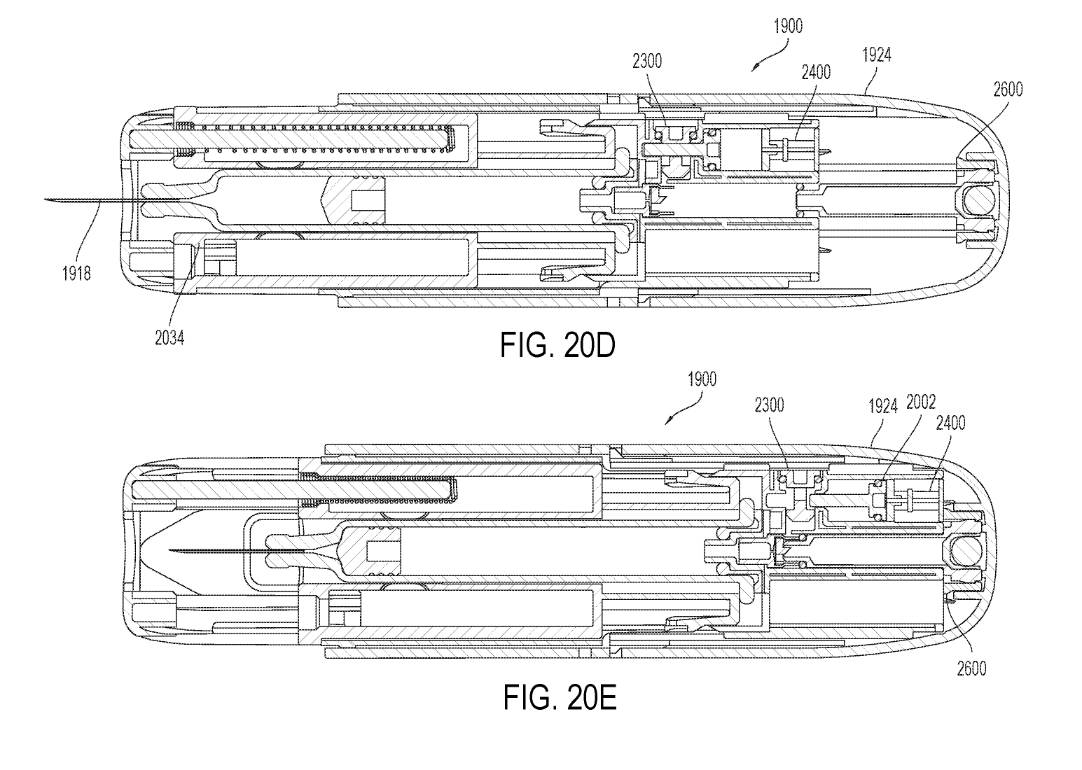

FIGS. 19A-20E illustrate another exemplary embodiment of an autoinjector 1900 in accordance with the subject disclosure. This embodiment of the autoinjector operates and includes features substantially as disclosed for the above embodiment, except as specifically discussed hereinafter. Additionally, the present exemplary autoinjector embodiment does not include a plunger or cantilever as featured in the above embodiment. FIGS. 19A-E illustrate a main body housing 1924 of a housing 1912. The main body housing includes a visual indicator window 1948 positioned about a proximal end of the housing and along a major face or anterior face of the autoinjector. As further described below, the window provides a visual indication as to the state of operation of the autoinjector. The window is activated, e.g., by displaying a visually identifiable feature 2602 therethrough (FIGS. 19B and 19C), based upon the state of operation of the autoinjector. While the visual indicator window can include a single window for indicating respective states of the autoinjector, the visual indicator window can alternatively include a plurality of windows e.g., 2, 3, 4, 5 or more than 5 windows, for indicating any of a series of states or changes of state that the autoinjector progresses through, from an unused ready-for-use state to a finished/spent retracted state. Further, the visual indicator window can be configured to include any type of indicator visible through the window(s), e.g., color coded indicators and various symbols or markings to associate with and identify the various states of the autoinjector, described in further detail below.

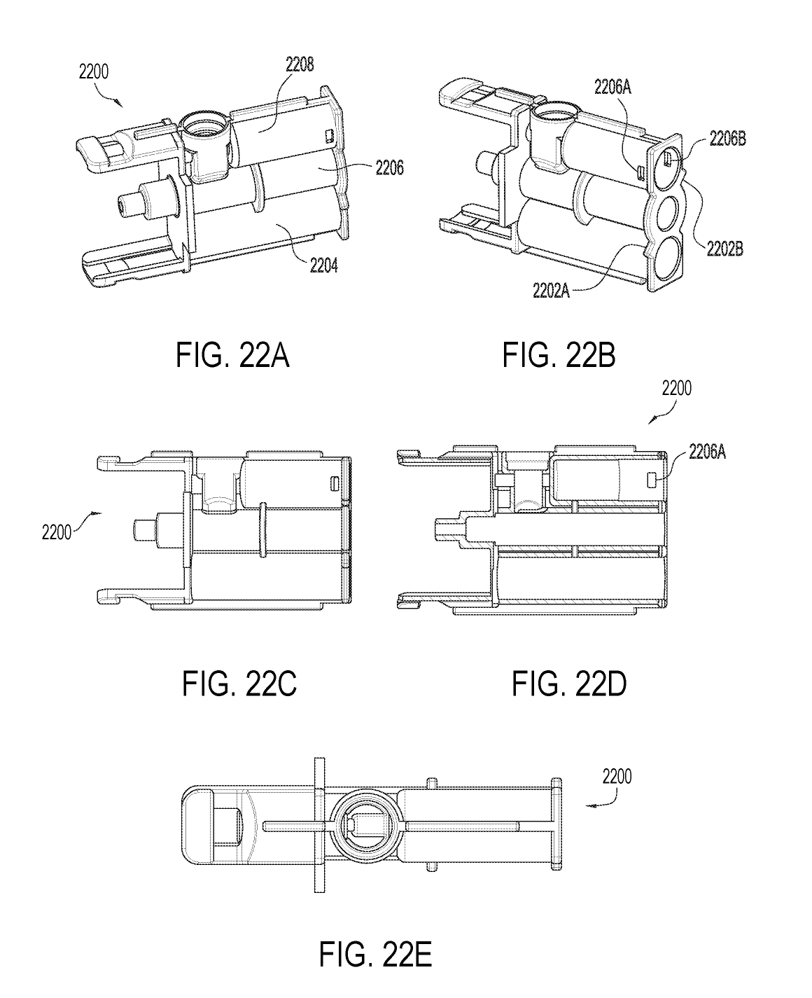

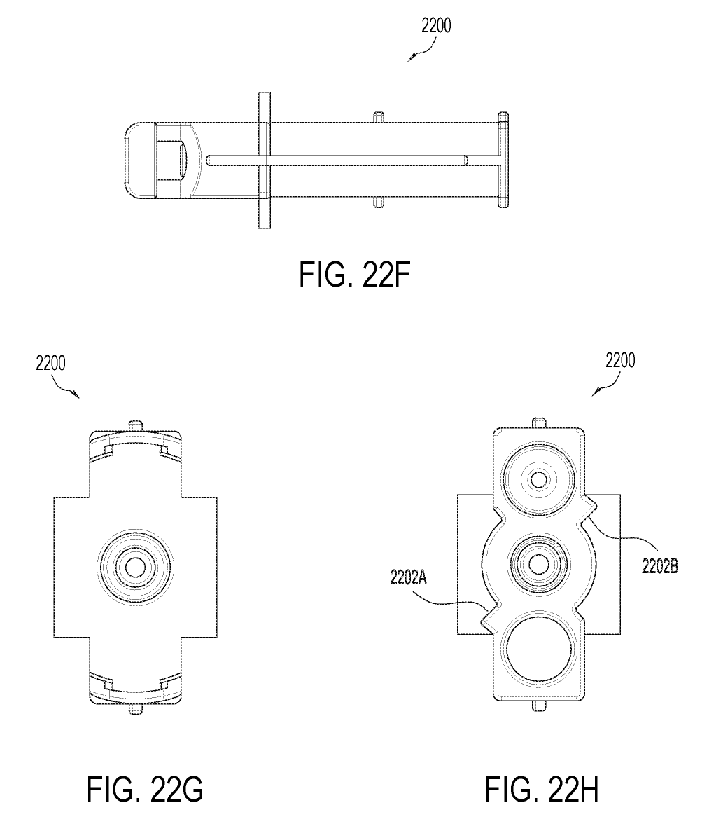

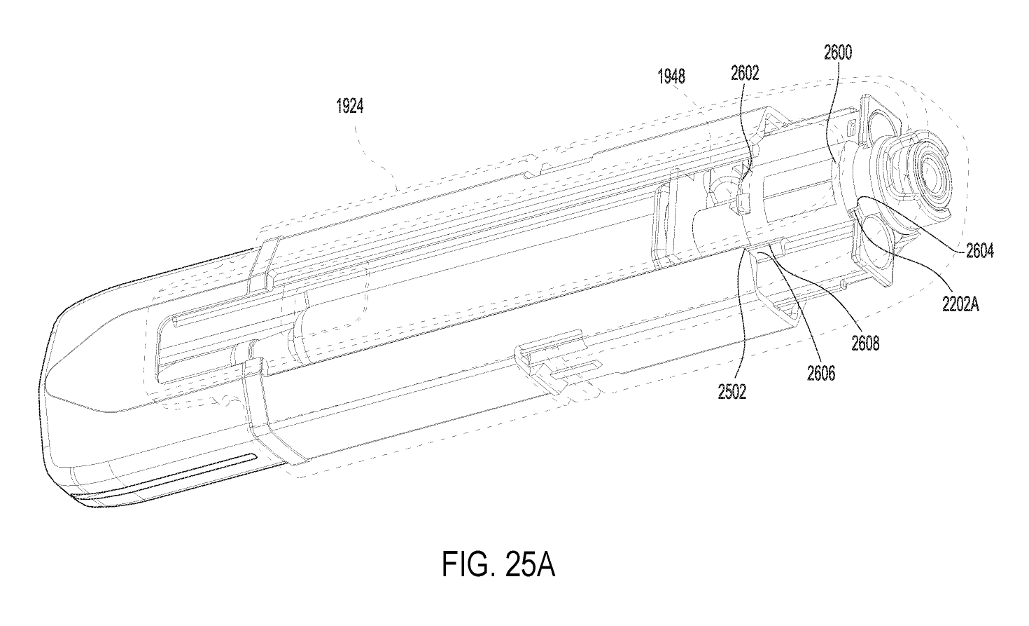

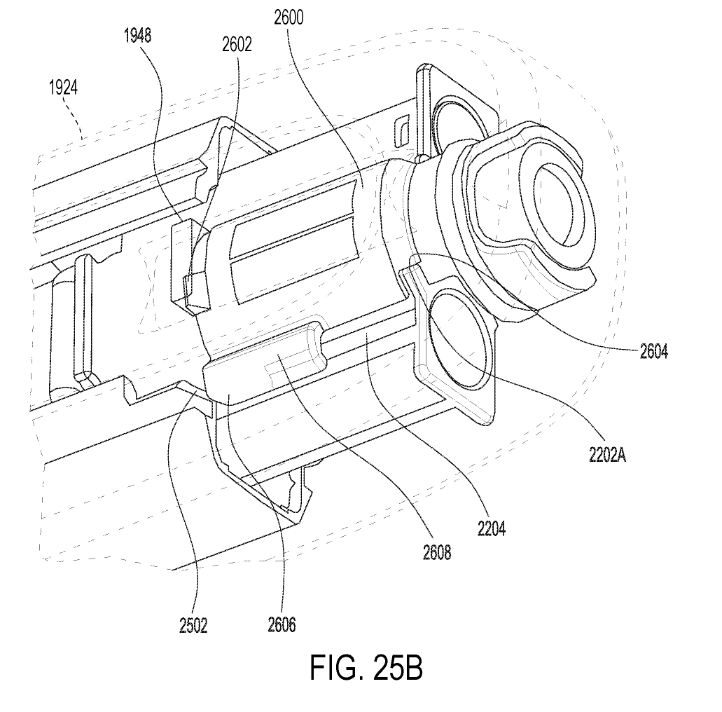

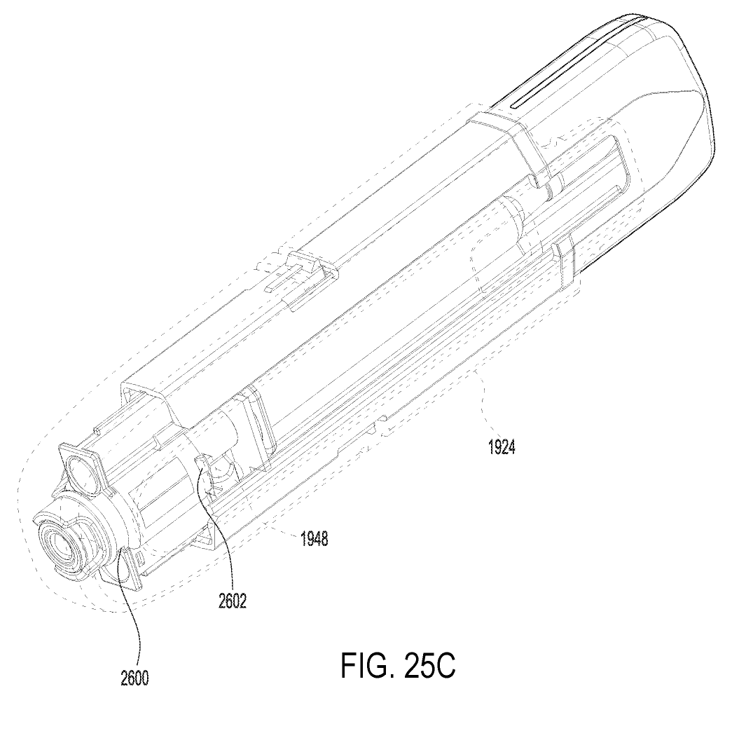

FIGS. 22A-H illustrate an exemplary embodiment of an actuator body 2200 applicable to the present autoinjector embodiment. The actuator body includes one or more stops 2202A, 2202B, a first housing 2206, a second housing 2208, and a third housing 2204. The first and second housings 2206, 2208 are configured as discussed above for the first and second housings 916, 918, respectively. The stops can be configured as tabs. The third housing is configured as a substantially cylindrical housing. However, the third housing can alternatively be configured as a non-cylindrical housing, such as a housing having a square, triangular, oval, or rectangular cross-sectional profile. The stops and the third housing are configured to bias an activator 2600 to rotate about a longitudinal axis of the actuator body, as described in further detail below with regard to FIGS. 25A-C.

The actuator body also includes one or more slots 2206A, 2206B about the proximal end of the first housing of the actuator body. The slots are configured for cooperating engagement with catches 2402A, 2402B of the fluid reservoir 2400, so as to mount the fluid reservoir in the proximal end of the first housing of the actuator body.

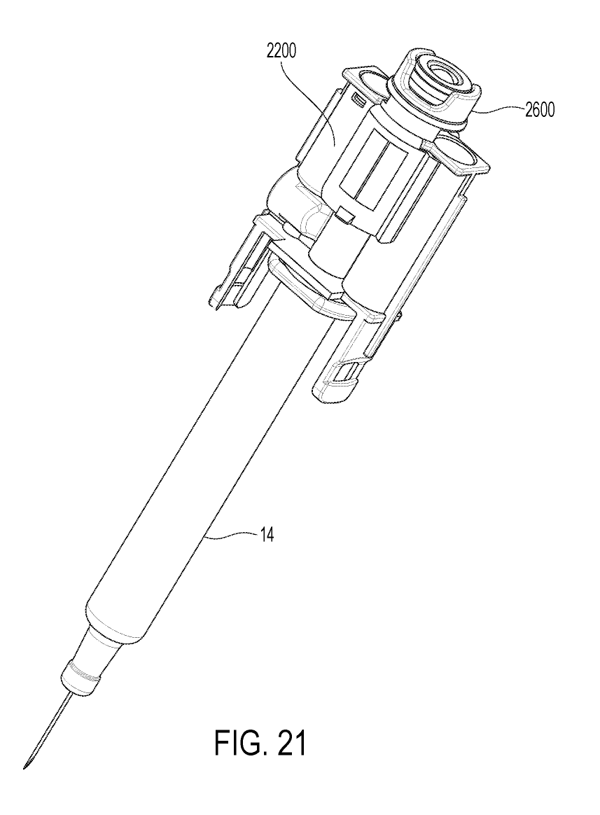

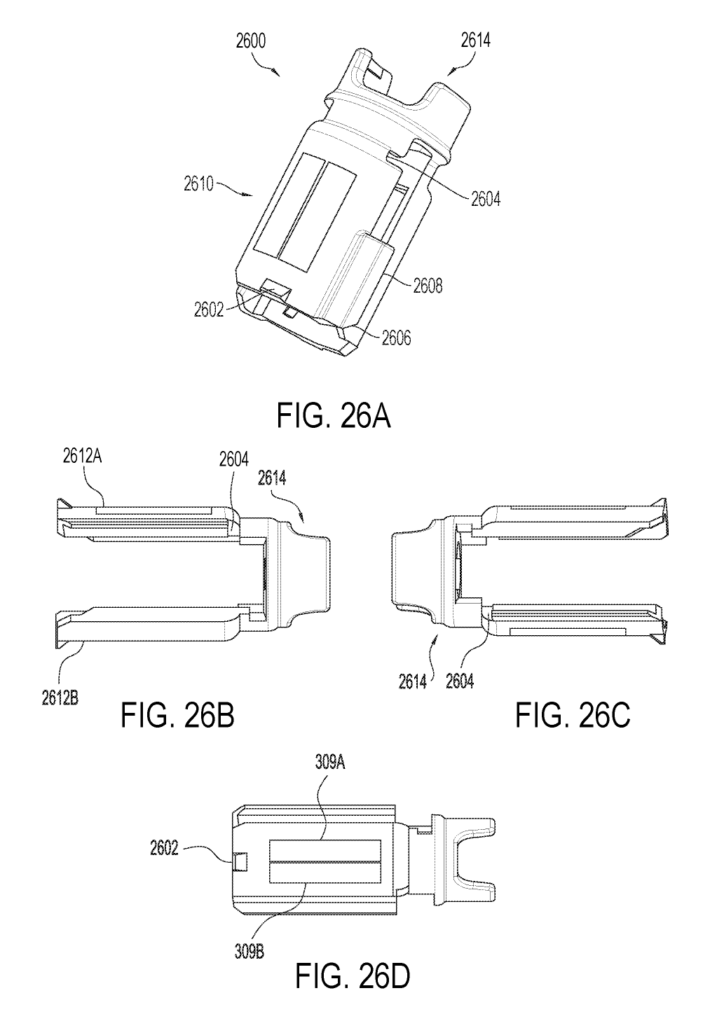

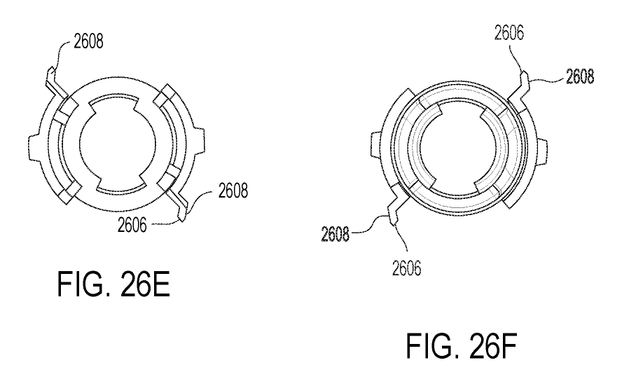

In accordance with an exemplary aspect of the present embodiment, the autoinjector 1900 includes the activator 2600 configured as best shown in FIGS. 21 and 26A-F. The activator is situated about a proximal end of the housing 1912 and receives the actuator assembly and canister. The activator has a substantially circular cap 2614 and a substantially tubular body 2610 including one or more arms 2612A, 2612B. The body can include one or more notches 2604 toward the proximal end of the activator. Although the activator can include two arms, the activator can alternatively include a single arm, or a plurality of arms, e.g. 3, 4, or more than 4 arms. Referring to FIG. 26D, the arms can have one or more visual indicators 309A, 309B, 2602.

The arms each have a fin 2608. Each fin is configured to be substantially curved and mounted toward a distal end of each arm. Each fin can additionally include an angled surface 2606.

The activator circumscribes the actuator assembly and is movable relative to the actuator assembly. In one exemplary aspect, the activator is rotatable about the actuator assembly. The activator is moveable relative to the actuator assembly between a first position such as an initial received or uncapped position (FIGS. 20A-B) and a second position (FIG. 20C), such as an activated position.

To facilitate rotation of the activator 2600, when the autoinjector shield is initially moved proximally (e.g., to an activated or armed state), the angled surface 2502 of the autoinjector shield abuts and engages the angled surface 2606 of the fin 2608, thereby camming and biasing the activator to rotate about the actuator toward the first housing 2206 of the actuator body. The rotation moves the notch 2604 of the activator away from the catch 2202A of the actuator body, thereby releasing and freeing the actuator assembly to be movable either proximally or distally relative to the autoinjector shield, as described in further detail below.

Referring to FIGS. 25A-C and 26A, initially in the first stage (e.g., an initial received or uncapped state), each notch 2604 of the activator is matingly and lockingly engaged with a corresponding stop 2202A, 2202B of the actuator body, as best shown in FIG. 25B. The fin 2608 of the activator also mates or engages with the third housing 2204 of the actuator. Accordingly, the locking engagement of the one or more notches 2604 with the stops 2202A, 2202B of the actuator body 2200 keeps the actuator body in place and prevents the actuator body from being freely movable either proximally or distally. The indicator 2602 is also positioned on the activator 2600 such that it is visible in a first position (FIG. 19B) relative to the window 1948 when the autoinjector 1900 is in the first stage.

When the user grasps the housing 1912 and depresses the distal end of the autoinjector to an injection site, the autoinjector transitions from the first stage to the second stage (e.g., from an initial received state to an activated or armed state) and the autoinjector shield is driven proximally toward the activator. The autoinjector shield cams and biases the activator 2600 to rotate about the actuator body 2200. The rotation disengages the notch 2604 from the catches 2202A, 2202B of the actuator body, allowing the actuator body to be freely movable. Consequently, the biasing force from the retraction biasing member biases the cradle and actuator body proximally (e.g., until the actuator body abuts a distal end of the cap 2614 of the activator). The proximal motion of the actuator body drives the regulator to pierce the canister seal to begin the injection. The rotation also moves the visual indicator 2602 of the activator to become visible in a second position (FIG. 19C) relative to the window 1948. Furthermore, once disengaged from the activator, the actuator body also becomes movable distally. Once the collective pressures throughout the actuator assembly build up from the pressurized gas released from the canister, the distal motion of the actuator body drives the syringe needle distally and begins the desired injection and retraction, as discussed in further detail below in connection with FIGS. 15A-18.

In an exemplary aspect of the present embodiment, the visual indicators 309A, 309B each correspond to a particular state of operation of the autoinjector and can be visible through a window 1950 (FIG. 19D) that is sized and shaped appropriately so as to display the visual indicator. As shown in FIG. 19D, a non-limiting exemplary window can be a rectangular window that is positioned about a proximal end of the housing and along a major face or anterior face of the autoinjector that is substantially the size and shape of the visual indicators 309A, 309B. The relative size of the visual indicators 309A, 309B compared to the visual indicator 2602 allows for a straightforward way to communicate more information about a current state of the autoinjector's operation, such as “Ready to use,” or “Injection in progress.”

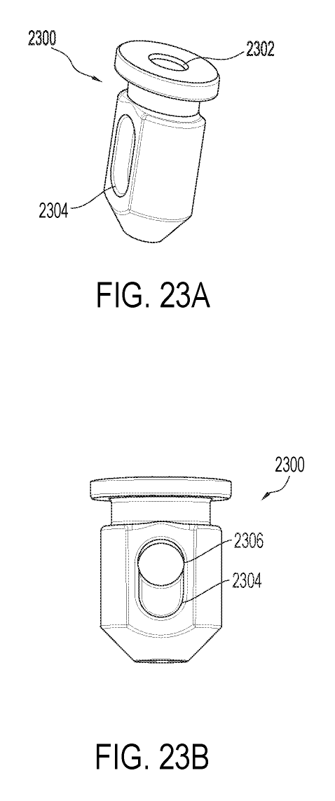

FIGS. 23A-B illustrate an alternative exemplary embodiment of a second timing member 2300 applicable to the present autoinjector embodiment. FIG. 23A illustrates that the second timing member is configured with a vent aperture 2302 in the increased width portion. The vent aperture is configured to facilitate venting of the pressurized gas to the exterior of the actuator assembly. FIGS. 23A-B illustrate that the second timing member also includes a through hole 2306 and a substantially elliptical or oval aperture 2304 that circumscribes the through hole. The through hole 2306 is substantially equivalent to the through hole 1106 discussed above (FIG. 11B). In an exemplary aspect, the oval aperture extends partially through either side of the reduced width portion.

Advantageously, in an exemplary aspect of the present embodiment, the oval aperture can provide for a sequenced retraction of the syringe needle. In a first phase of the sequenced retraction, the force of the pressurized gas drives the first timing member 2002 (FIGS. 20A-E) proximally past the through hole 2306, but not past the oval aperture. The force of the pressurized gas drives the second timing member laterally, such that the first timing member lockingly engages with the oval aperture. In a subsequent phase of the sequenced retraction, the force of the pressurized gas continues to drive the first timing member proximally so that the second timing member becomes free of locking engagement with the first timing member by the oval aperture.

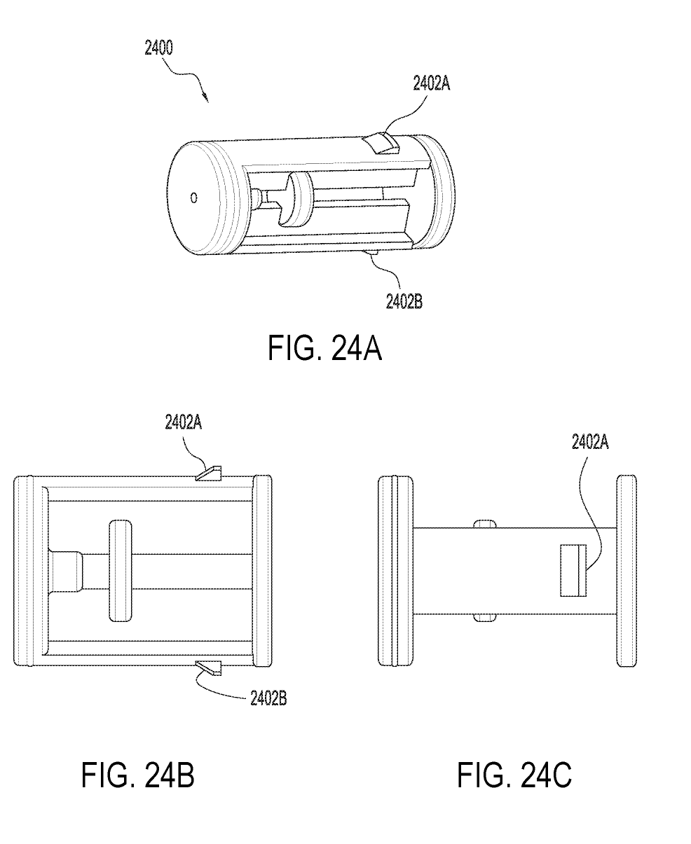

FIGS. 24A-C illustrate an alternative embodiment of a fluid reservoir or delay plug 2400 applicable to the present autoinjector embodiment. The fluid reservoir includes catches 2402A, 2402B toward the proximal end of the fluid reservoir. The catches can be configured as tabs. The catches are configured to engage with the slots 2206A, 2206B of the actuator body, so as to mount the fluid reservoir in the proximal end of the actuator body.

Operation of the autoinjector 10 can be broken down into several operational states. The states include a first, second, third, and fourth stage. Non-limiting exemplary operational states can include the first stage corresponding to an initial received or uncapped state (FIGS. 15A-B), the second stage corresponding to an activated, armed, or ready-to-use state (FIGS. 16A-B), the third stage corresponding to an injection end-of-dose state (FIGS. 17A-B), and the fourth stage corresponding to a retracted state (FIGS. 18A-B). FIGS. 15A-B illustrate the actuator body 900 in the first stage or initial received or uncapped state, and FIGS. 16A-B illustrate the actuator body in the second stage or activated, armed, or ready-to-use state.

In operation, a user, e.g., grasps the autoinjector 10, e.g., with his right hand and pulls off the removable housing section 26 with his left hand. Then, to make an injection, while grasping the main body housing 24, the user depresses the distal end of the autoinjector to the targeted injection site. This depression moves the autoinjector to its second stage, e.g., the activated, armed, or ready-to-use state. As best shown in FIG. 20C, the depression drives the autoinjector shield 100 proximally relative to the housing 12. The cooperating tabs 302A, 302B on the autoinjector shield move from their engagement with the distal cooperating slots 110A, 110B on the housing into the proximal cooperating slots.

In accordance with an exemplary aspect of the present embodiment, the cantilever 808 (FIGS. 8, 9A) of the actuator body 900 is initially engaged with the extended slats 108 of the autoinjector shield 100 (FIG. 4), which holds the autoinjector shield in the initial position until biased proximally to begin an injection. The cantilever deflects so as to disengage from the autoinjector shield, thereby freeing the actuator assembly.

As the autoinjector shield continues its proximal motion, the actuator assembly 1500 is freed to be movable either proximally or distally relative to the autoinjector shield, e.g., from the second stage to the third stage. Once the actuator assembly is disengaged, the force from the retraction biasing member 42 biases the cradle 34 and the actuator assembly proximally, thereby driving the regulator proximally toward the canister, so as to pierce the canister and release the pressurized gas therein.

Operation of the third and fourth stages is hereinafter discussed with respect to autoinjector 1900. Operation of the autoinjector 1900 operates similarly to autoinjector 10, but the activator 2600 (FIG. 26A) rotates so as to disengage from the actuator assembly, thereby freeing the actuator assembly. As best shown in FIG. 20D, the autoinjector transitions to a third stage in which the autoinjector performs the desired injection. A force from the pressurized gas drives the actuator assembly distally relative to the autoinjector shield. The distal motion of the actuator assembly drives the cradle 1934 distally, thereby extending the syringe needle 1918 past the autoinjector shield to dispense the desired dosage of medicament. The distal motion of the cradle also elongates the retraction biasing member 1942 against the pin 1937.

In accordance with an exemplary aspect, the exemplary autoinjector embodiments discussed herein are operable to administer intramuscular, subcutaneous, or other injections.

As best shown in FIG. 20E, the autoinjector transitions to a fourth stage in which the cradle 1934 and the actuator assembly retract the syringe needle 1918, so as to finish the desired injection. Upon completion of an injection by the autoinjector, or a complete venting of the pressurized gas within the actuator assembly, the retraction biasing member biases the cradle and actuator assembly in the proximal direction, thereby retracting the syringe fully within the autoinjector shield.

With reference to FIGS. 15A-B and autoinjector 10, in the first stage the regulator 1400 is spaced from the canister 204. In other words, the canister is in a sealed state with the pressurized driver at a first pressure P1, and the flow regulator 228 is not in fluid communication with the canister or a flow of pressurized driver provided by the canister. As described above, the pressure P1 is at a vapor pressure for a liquefied gas, which can range for non-limiting exemplary liquefied gases at room temperature from about 17 psi for isobutane, about 63 psi for dimethyl ether (DME), about 71 psi for 1,1,1,2-Tetrafluoroethane (R134a), about 108 psi for propane, about 760 psi for nitrous oxide, to about 850 psi for carbon dioxide.

With reference to FIGS. 16A-B, in the second stage, the proximal motion of the autoinjector shield deflects the angled surface of the cantilever so as to disengage the actuator assembly 1500 from the autoinjector shield. Consequently, the actuator assembly becomes freely movable proximally or distally. The force from the retraction biasing member 42 biases the actuator body 900 proximally toward the canister 204 such that the regulator 1400 pierces the seal 218 of the canister. Accordingly, in the second stage, the regulator is in fluid communication with the canister for receiving a flow of gas from the canister. Gas released from the canister flows through the flow regulator 228 and out through the open distal end 908 of the actuator body 900. Alternatively expressed, the regulator engages the canister to allow fluid communication of a flow of gas from the canister with the flow regulator 228.

As the pressurized gas exits the canister and the collective pressures build in the actuator assembly 1500, the actuator body 900 moves distally relative to the autoinjector shield and canister. The collective pressures throughout the actuator assembly thereby drive the actuator assembly and cradle 34 distally to extend the syringe needle and inject the medicament.

Upon actuation, once the gas exits the canister, the pressurized gas is at pressure P1 in the space proximal to the regulator defined, e.g., by the proximal end of the regulator, the interior walls of the proximal end of the regulator housing 230, and the distal end 214 of the canister.

The regulator housing 230 is configured to provide a substantially constant rate, or flow, of pressurized gas exiting the outlet 1404 via the flow regulator 228 into the smaller diameter receptacle region 910. The regulator or flow regulator does not regulate the pressure of the driver passing therethrough. Instead, the regulator housing advantageously provides a substantially constant flow of gas out through the flow regulator 228 regardless of the pressure P1 exiting the canister 204.

The pressurized gas flows from the canister 204 in the larger diameter receptacle region 932 through the open distal end 908 towards the plunger 1300 and/or the stopper 25. As pressurized driver is released from the canister 204 in the larger diameter receptacle region 932, pressure builds up both in the larger diameter receptacle region and in the smaller diameter receptacle region 910. The pressurized gas then 1) exits the smaller diameter receptacle region out the open distal end 908 of the actuator body 900 and 2) exits the larger diameter receptacle region through the flow passageway 922 to the relief valve assembly 902.

When the pressurized gas flow exits the flow regulator 228, the pressurized gas enters a space, e.g., defined by the interior walls of the distal end of the regulator housing, the interior walls of the smaller diameter receptacle region 910, the interior walls of the neck 944, and the interior walls of the plunger 1300. The pressurized gas is at a pressure P2 that is less than pressure P1. As described earlier, one advantage of the exemplary autoinjector embodiments is that, by appropriate sizing of the flow regulator 228, the regulator housing regulates the flow of gas and thereby reduces the pressure of the driver in this space, denoted by P2, such that the pressure is below P1 but above the pressure necessary to move the plunger 1300 and/or the stopper 25. This causes the driver and/or the plunger to have the effect of a conventional syringe plunger and drive the stopper distally towards the needle 18 and, consequently, drive the medicament distally out through the needle 18. The pressure P2 of the pressurized gas flow entering the smaller diameter receptacle region via the flow regulator is less than the initial pressure P1 of the gas in the canister. By way of non-limiting example, the pressure P2 is significantly less than P1, ranging from about 10 to about 20 psi, or about 1%, 5%, 10%, 15%, 20%, 25%, 30%, 40%, or 50% of the pressure P1.

In accordance with an exemplary aspect, the drive chamber 1606 is defined, e.g., by the open distal end 908 of the actuator body and the interior of the plunger 1300 as shown in FIG. 16B. That is, the flow of gas imparts a force to drive the plunger distally which in turn drives the stopper 25 distally within the syringe barrel 20. In accordance with another exemplary aspect, the plunger can be omitted and the drive chamber is defined by the open distal end of the actuator body, the interior of the syringe barrel, and the proximal end of the stopper. That is, the flow of gas imparts a force that acts on the stopper 25 directly to drive the stopper distally within the syringe barrel.

As the stopper 25 moves distally down the length of the syringe barrel 20, the volume of the drive chamber 1606 increases as the flow of gas continuously enters, thereby maintaining the pressure P2 at a substantially constant pressure throughout the space defined by the distal end of the regulator housing 230, the interior walls of the smaller diameter receptacle region 910, the interior walls of the neck 944, and the drive chamber 1606. The pressure P2 remains substantially constant until the stopper 25 reaches a predetermined end of the desired dose, as described in further detail below regarding the third position.

The collective effect of how the pressures are supplied by the liquefied gas within the autoinjector 10 provides an adaptive force for applying to the stopper 25 to drive the medicament from the syringe or medicine cartridge chamber 14. The adaptive force increases or decreases based upon a change in speed of travel of the stopper or a change in force required to move the stopper. For example, as a flow of gas provided by the liquefied gas exits the canister 204, it applies an adaptive force to the stopper in a controlled manner such that: i) the adaptive force will be constant at a constant injection rate (e.g., a substantially constant rate of travel of the stopper along the syringe barrel), ii) the adaptive force will increase, if the injection rate slows to below the constant injection rate (e.g., due to stiction between the stopper and the interior walls of the syringe barrel 20 or medicine cartridge chamber), and iii) the adaptive force will decrease, as the injection rate increases from below the constant injection rate towards the constant injection rate (e.g., due to excess lube between the stopper and the interior walls of the syringe barrel or medicine cartridge chamber).

As described above, the pressurized gas also flows into the relief valve assembly 902 after exiting the canister 204. The flow of gas travels up through the flow passageway 922 and into the first and second internal cavities 924, 926. Initially the first timing member 1000 is in the primary position that locks the second timing member 1100 in the first position. In this primary position, the first timing member extends through the through hole 1106 of the second timing member and the second timing member is directly engaged with the first timing member while the second timing member seals the first opening 912 of the relief valve assembly. The flow of gas exiting the canister exerts a force P1 at the inlet of flow passage 922 that can initially limit the pressure exerted on the first timing member 1000. In other words, initially the force exerted on the relief valve assembly can be insufficient to overcome the countering force of the damping element engaging the first timing member and the sealing engagement of the sealing members 1502, 1504 due to the flow restriction effect of the flow passage 922. However, over a brief period of time, the pressure exerted on the first timing member reaches P1, thereby moving the first timing member to its secondary position. Alternatively, the sizing of the flow passage 922 can be sufficiently sized so as not to limit the pressure, and a pressure of P1 is instantaneously exerted therethrough. The pressure P1 exerts a force that pushes the first timing member 1000 proximally toward the second opening 914 and pushes the second timing member 1100 laterally toward the first opening 912. Advantageously, the delayed exhaust of the pressurized driver based on the first and second timing members allows precise and configurable control over the timing and speed of the fourth stage, e.g., retracting the syringe needle 18 relative to the autoinjector shield 100.

However, the damping element 232 operates to limit the rate and timing at which the first timing member is pushed fully into the reservoir chamber 920 and toward the second opening. Specifically, the first timing member in conjunction with the reservoir chamber is configured to reach its secondary position at a time sufficiently beyond the time needed to complete an injection of the medicament from the syringe. In other words, the first timing member reaches its secondary position a predetermined amount of time after the injection is completed. For example, if the injection is completed 1 second after the activation/armed/ready-to-use stage, the first timing member is configured to reach its secondary position about 2-5 seconds after activation.

The damping element can be a viscous fluid. The viscous fluid can be any known viscous fluid that can serve as a countering force or frictional force against the motion of the first timing member. Examples of damping elements include, but are not limited to, damping grease, Society of Automotive Engineers (SAE) grade 10, 30, 50, or 70 oils, and the like. The choice of damping element is used to control the timing and/or speed of the first timing member reaching its secondary position.

With reference to FIGS. 17A-B, in the third stage upon reaching the end of the desired injection dose, the volume of the space defined, e.g., by the interior walls of the distal end of the flow regulator, the interior walls of the smaller diameter receptacle region 910, the interior walls of the neck 944, and the interior walls of the drive chamber 1606 remains fixed. However, the pressurized gas continues to exit the canister at a pressure P1, thereby increasing in pressure to P3+, and up to a pressure of P1 until the relief valve assembly vents.

In accordance with an exemplary aspect, during the third stage the desired dose may be administered as a substantially full dose of medicament, whereby the stopper bottoms out at the distal end of the syringe barrel 20. In accordance with another exemplary aspect, the desired dose may be administered as a precise dosing of only part of the syringe contents whereby the plunger 1300 reaches the end of its stroke before the stopper reaches the distal end of the syringe barrel.

With reference to FIG. 18, in the fourth stage the second timing member 1100 has been pushed laterally from the relief valve chamber 940, thereby allowing the cradle 34 to retract within the housing. The proximal end of the first timing member abuts the distal end of the fluid reservoir. When the first timing member is moved far enough into the reservoir chamber 920, the first timing member exits the through hole 1106 of the second timing member. Once the second timing member is free of locking engagement with the first timing member, the influence of the force on the second timing member moves the second timing member laterally such that the pressurized gas can vent to an exterior of the actuator assembly.

In an exemplary aspect of the present embodiment, the first opening 912 of the actuator body may be configured to generate an audible sound, e.g., a whistle, so as to emit an audible indicator, as the pressurized gas is released or vented, similar to a conventional air whistle. In further exemplary aspects, the exterior of the actuator assembly where pressurized gas vents can be confined to the autoinjector, so as to avoid venting the pressurized gas to an exterior of the autoinjector. In still further exemplary aspects, the force of the venting gas can be used to provide some other form of action or motion, e.g., indication, insertion, retraction, timing, delay, or another ancillary function of the autoinjector.

As a consequence of the pressurized gas venting to the exterior of the actuator assembly, the collective pressures in the actuator assembly are reduced to substantially zero and the proximally directed retraction force from the retraction biasing member 42 drives the cradle 34 and the syringe 14 proximally relative to the autoinjector shield 100, thus retracting the syringe back into the autoinjector 10. Advantageously, the speed and timing of this retraction can be precisely controlled based on appropriate selection of a configuration of the actuator assembly, first and second timing member, and/or a damping element having appropriate viscosity to slow the proximal movement of the first timing member 1000.

The various exemplary embodiments of the autoinjector discussed herein provide numerous advantages over conventional autoinjector devices. For example, the present autoinjector uses a relief valve assembly that interfaces with several components via timing members, a damping element, and a fluid reservoir to provide precise and configurable control over the timing and speed of retraction following an injection. One advantage of using a relief valve assembly is the ability to leverage a second or ancillary function of the force provided by the driver, to provide another form of action such as delaying or otherwise configuring the timing of retraction.

Another advantage of the exemplary autoinjector embodiments is use of an intermediate member, e.g., plunger 1300, that provides for precise and configurable dosing of only part of the syringe contents. For example, the present autoinjector uses the intermediate member in combination with dosage stops on the actuator body to provide a dose stop feature during use, i.e., partial dosage applications.

A further advantage of the exemplary autoinjector embodiments is use of a rotating drum. For example, the present autoinjector uses a rotating drum, e.g., activator 2600, that interfaces with several components via tracks, stops, and cams, to control the sequence of operational states during use. This provides several benefits over conventional autoinjector mechanisms, especially in a marketplace looking for premium features on a low-cost disposable device.

One advantage of using an activator is that it allows greater control of the device operation and features, without adding undue complexity and cost. The activator provides for a highly configurable sequence of events with precise control of each operational state. The activator is also able to integrate features that would be impractical and/or expensive with conventional autoinjector mechanisms. For example, certain advantageous features integrated into the activator concept include status indicators and locked syringe positions.

Further advantages of the exemplary autoinjector embodiments include status indicators. An advantage of the activator approach is that it allows for a straightforward way to communicate the current state of the autoinjector's operation, such as “Ready to activate,” “Injection in progress,” or “Injection completed.” With the design of the exemplary embodiments, components such as the shield, syringe, and actuator have physical interfaces with the activator so that the activator itself can be used to indicate to the user the current state of operation in a convenient and straightforward way, using e.g., windows and visible markings, or switches and electronic indicators.

A yet further advantage of the exemplary autoinjector embodiments is its locked syringe positions. Typical conventional autoinjectors have mechanisms that do not allow for securely locking the syringe in position, and instead rely on spring forces to keep it in position during handling and after usage. This may allow the syringe and needle to move outwardly during shocks or mishandling, possibly causing needle stick injuries. In contrast, the activator of the exemplary embodiments allows for a securely locked needle in all positions.

Another advantage of the exemplary autoinjector embodiments is auto retraction of the needle into the autoinjector body. In contrast, conventional autoinjectors use a spring-loaded shield to cover the needle as the device is removed from the injection site. Thus, in combination with status indicators, auto retraction simplifies operational steps required of the user in order to ensure an effective and safe injection.

A still further advantage of the exemplary autoinjector embodiments is its no-force shield. Typical conventional autoinjectors require the user to maintain force against the injection site during injection to keep the needle shield's spring compressed until the injection is complete, at which time the pressure is removed and the shield extends and locks in place as the device is withdrawn. This is supposed to be a simple and effective approach, but the user's grip may slip or be repositioned while the injection is occurring. As a result, the device may be pushed away from the injection site by the shield's spring, thus locking the shield over the needle and injecting the remaining contents into air. This results in an underdose which is not only a nuisance but in some cases can result in a fatal error. The design of the exemplary autoinjector embodiments reduces this possibility since during retraction the syringe moves relative to the shield and the pressurized gas itself provides the force for needle insertion and injection.

It will be appreciated by those skilled in the art that changes could be made to the exemplary embodiments described above without departing from the broad inventive concept thereof. For example, additional components, types of timing pins, types of plungers, types of regulators, or visual indicators can be added or used with the autoinjector. It is to be understood, therefore, that the present exemplary embodiments of the autoinjector and its various components are not limited to the particular exemplary embodiments disclosed, but it is intended to cover modifications within the spirit and scope of the present invention as defined by the claims.

I/We claim:

1. A power pack for an autoinjector comprising:
an actuator assembly for mounting to an autoinjector, the actuator assembly including:
an actuator body having:
a receptacle for receiving a canister,
an open distal end in fluid communication with the receptacle, and
a channel in fluid communication with the receptacle, and
a relief valve assembly mounted to the actuator body, the relief valve assembly including:
a first timing member movable between a primary position and a secondary position under the influence of a force, and
a second timing member movable between a first position directly engaged with the first timing member and a second position spaced from the first timing member.

2. The power pack of claim 1, further comprising a canister having a pressurized driver mounted within the receptacle.

3. The power pack of claim 1, further comprising a regulator mounted within the receptacle.

4. The power pack of claim 1, wherein the actuator body further includes a recess for receiving the relief valve assembly, and wherein the recess is in fluid communication with the channel.

5. The power pack of claim 1, wherein when in the primary position, the first timing member locks the second timing member in the first position.

6. The power pack of claim 1, wherein the relief valve assembly houses a damping element engaging the first timing member.

7. The power pack of claim 6, wherein the damping element is a viscous fluid.

8. The power pack of claim 6, wherein the relief valve assembly further comprises a reservoir chamber housing the damping element.

9. An autoinjector comprising:
a housing;
the power pack of claim 1 mounted within the housing; and
a medicament container mounted within the housing and operatively connected to the power pack and in fluid communication with the open distal end of the actuator body.

10. A power pack for an autoinjector comprising:
an actuator assembly for mounting to an autoinjector, the actuator assembly including:
an actuator body having:
a first housing for receiving a canister, the first housing having an open distal end,
a second housing in fluid communication with the first housing, the second housing including a first opening in fluid communication with an exterior of the actuator body and a second opening in fluid communication with the exterior of the actuator body and spaced from the first opening.

11. The power pack of claim 10, further comprising a canister having a pressurized driver mounted within the first housing.

12. The power pack of claim 10, further comprising a regulator mounted within the first housing.

13. The power pack of claim 10, further comprising a relief valve assembly mounted within the second housing, the relief valve assembly including:
a first timing member movable between a primary position and a secondary position, and
a second timing member movable between a first position sealing the first opening and a second position spaced from the first opening.

14. The power pack of claim 13, wherein the relief valve assembly further comprises a reservoir chamber housing a damping element engaging the first timing member.

15. The power pack of claim 14, wherein the damping element is a viscous fluid.

16. The power pack of claim 14, wherein the reservoir chamber housing sealingly engages the second opening.

17. The power pack of claim 13, wherein the first timing member moves along an axis substantially transverse to an axis along which the second timing member moves.

18. The power pack of claim 13, wherein when in the primary position, the first timing members locks the second timing member in the first position.

19. An autoinjector comprising:
a housing;
the power pack of claim 10 mounted within the housing; and
a medicament container mounted within the housing and operatively connected to the power pack and in fluid communication with the open distal end of the actuator body.
